(19) United States
(12) Patent Application Publication
(10) Pub. No.: US 2026/0027200 A1
(43) Pub. Date: Jan. 29, 2026

(54) Poxvirus mRNA Vaccine and Use

(71) Applicants: YITHER BIOTECH CO., LTD, Shanghai (CN); AB & B BIO-TECH CO., LTD. JS, Jiangsu (CN)

(72) Inventors: Zhihua LIU, Shanghai (CN); Yelin XIONG, Shanghai (CN); Caixia SU, Shanghai (CN); Youcai AN, Jiangsu (CN)

(21) Appl. No.: 19/469,683

(22) PCT Filed: Mar. 7, 2024
(86) PCT No.: PCT/CN2024/080492

§ 371 Date: Sep. 26, 2025

(30) Foreign Application Priority Data
Mar. 13, 2023 (CN) 202310244097.X

(51) Int. Cl. A61K 39/285; A61K 9/1271; A61K 9/51; A61K 39/00; A61P 31/20; B82Y 5/00; C12N 7/00

(52) U.S. Cl. CPC A61K 39/285; A61K 9/1271; A61K 9/5123; A61P 31/20; C12N 7/00; A61K 2039/53; A61K 2039/55555; A61K 2039/575; B82Y 5/00; C12N 2710/24134; C12N 2830/50

(57) ABSTRACT

Provided are a poxvirus mRNA vaccine and a use. Specifically, provided is an isolated mRNA molecule, comprising a coding region, the coding region coding a target protein, and the target protein comprising the following four proteins: vaccinia virus surface antigens A27, L1, A33, and B5, wherein every two adjacent proteins are independently directly linked or linked by identical or different linkers. Animal immunization results show that the mRNA molecule has good candidate vaccine potential and provides an innovative idea for the development of poxvirus vaccines.

### TECHNICAL FIELD

The present invention belongs to the field of biomedicine and relates to a poxvirus mRNA vaccine and use.

### BACKGROUND ART

Poxviruses are enveloped DNA viruses and are classified with Orthopoxvirus genus of Poxviridae family; some poxviruses can infect humans, causing serious diseases and even threatening human life; for example, there are four orthopoxviruses that can infect humans: variola virus (VARV), monkeypox virus (MPXV), cowpox virus (CPXV) and vaccinia virus (VACV). The cross protection provided by attenuated vaccinia virus vaccination has eliminated the variola virus, which has caused many pandemics in human history, from the human population, proving that vaccines are one of the most effective means of preventing and controlling viral infection and transmission. At present, only a few countries in the world have variola vaccines in reserve to deal with variola or similar orthopoxvirus epidemics; the frequent occurrence of monkeypox virus epidemics has once again attracted attention to the research and development of human poxvirus vaccines.

Given safety concerns and production challenges, increasing emphasis is being placed on development of genetically engineered poxvirus vaccines to prevent variola or monkeypox virus infections. Vaccinia virus surface antigens A27, L1, A33 and B5 contain a large number of high-quality conserved neutralizing antibody epitopes, and have been proven to be candidate antigens for variola vaccines and novel monkeypox virus vaccines. By inducing high levels of cross-humoral and cellular immunity to provide in vivo protection, multi-antigen design can theoretically greatly reduce the risk of viral mutation escape.

As a new type of vaccine, mRNA vaccines have been proven to have the potential to induce good humoral and cellular immunity in animals and humans. The basic immunological mechanism is that after mRNA is delivered into cells, it can express original conformation antigens, induce cellular immunity through MHC-I/II class presentation, and induce certain humoral immunity through interaction with B cells. Compared with traditional vaccine preparation technology, the advantage of mRNA technology is that when sufficient antigen information is obtained, the concept verification and process development of vaccine prototype preparation can be shortened to several months, with extremely high yield and short production cycle, which cmi meet the vaccination and prevention and control needs of large-scale sudden epidemics.

Current genetically engineered candidate poxvirus vaccines (subunit/DNA) fail to induce high-level balanced humoral immunity and cellular immunity; and often require adjuvant compatibility and separate design of four antigens, which increases difficulty of process development.

At present, there is still a need to develop new poxvirus mRNA vaccines.

### Contents of the Present Invention

After in-depth research and creative work, the inventors constructed a poxvirus mRNA molecule by rationally designing A27, L1, A33 and B5 antigen combinations. Animal immunization results show that the mRNA molecule has good candidate vaccine potential, and an innovative idea is therefore provided for the development of poxvirus vaccines. The present invention is thus provided as follows:

One aspect of the present invention relates to a isolated mRNA molecule, which comprises a coding region, wherein the coding region encodes a target protein, and the target protein comprises the following four proteins:
vaccinia virus surface antigens A27, L1, A33 and B5;
wherein, two adjacent proteins are independently connected directly or connected via identical or different linkers.

In some embodiments of the present invention, in the isolated mRNA molecule:
A27 has an amino acid sequence as set forth in SEQ ID NO: 6,
L1 has an amino acid sequence as set forth in SEQ ID NO: 7,
A33 has an amino acid sequence as set forth in SEQ ID NO: 8. and/or
B5 has n amino acid sequence as set forth in SEQ ID NO: 9.

In some embodiments of the present invention, in the isolated mRNA molecule, A27, L1, A33 and B5 are arranged in sequence in the target protein;
preferably, A27, Linker, L1, Linker, A33, Linker and B5 are arranged in sequence in the target protein.

In some embodiments of the present invention, in the isolated mRNA molecule, the target protein is composed of A27, Linker, L1, Linker, A33, Linker and B5 arranged in sequence.

In some embodiments of the present invention, in the isolated mRNA molecule, the Linker has amino acid sequence as set forth in SEQ ID NO: 10.

In some embodiments of the present invention, in the isolated mRNA molecule, the target protein has an amino acid sequence a set forth in SEQ ID NO: 2.

In some embodiments of the present invention, in the isolated mRNA molecule, the coding region has a sequence as set forth in SEQ ID NO: 11.

In some embodiments of the present invention, the isolated mRNA molecule further comprises one or more selected from the following:
5′UTR, stop codon, 3′UTR and PolyA;
preferably, the isolated mRNA molecule comprises in sequence:
5′UTR, coding region, stop codon, 3′UTR and PolyA.

In some embodiments of the present invention, the isolated mRNA molecule consists of 5′UTR, coding region, stop codon, 3′UTR and PolyA.

In some embodiments of the present invention, in the isolated mRNA molecule:
5′UTR has a sequence as set forth in SEQ ID NO: 12.
the stop codon has a sequence of UGAUAA,
3′UTR has a sequence as set forth in SEQ ID NO: 13, and/or
PolyA has a sequence as set forth in SEQ ID NO: 14.

In some embodiments of the present invention, the isolated mRNA molecule has a sequence as set forth in SEQ ID NO: 1.

In the present invention, if not specifically stated, the 5′ end cap structure and Kozak belong to the 5′UTR sequence, that is, they are regarded as parts of the 5′UTR sequence.

Another aspect of the present invention relates to an isolated DNA molecule, which can transcribe the isolated mRNA molecule described in any one of the items of the present invention.

In some embodiments of the present invention, the isolated DNA molecule comprises a DNA fragment having a sequence as set forth in SEQ ID NO: 3 or SEQ ID NO: 4.

The inventors found that the protein expression level of the DNA fragment of the sequence as set forth in SEQ ID NO: 4 is higher in vivo or in vitro than that of the DNA fragment of the sequence as set forth in SEQ ID NO: 3.

In some embodiments of the present invention, the isolated DNA molecule further comprises a promoter located upstream, preferably a T7 promoter.

In some embodiments of the present invention, the isolated DNA molecule has a sequence as set forth in SEQ ID NO: 5.

Another aspect of the present invention relates to a recombinant plasmid, which comprises the isolated DNA molecule as described in any one of the items of the present invention.

Another aspect of the present invention relates to a recombinant host cell, which comprises the isolated DNA molecule as described in my one of the items of the present invention or the recombinant plasmid as described in the present invention.

Another aspect of the present invention relate to an mRNA vaccine preparation, which comprises the isolated mRNA molecule as described in any one of the items of the present invention, and an mRNA vaccine vector;
preferably, the mRNA vaccine vector is a lipid nanoparticle or a polymer nanoparticle;
preferably, the lipid nanoparticle;
preferably, the mRNA vaccine preparation has an average particle size of 50-100 nm, 70-90 nm, 80-90 nm or 85 nm.

In some embodiments of the present invention, the mRNA vaccine preparation is a vaccine preparation for preventing or treating variola or monkeypox virus infection.

The vaccine preparation may comprise one or more vaccine carriers or excipients. The carriers or excipients for the vaccine are well known in the art (see, for example, Remington's Pharmaceutical Sciences. Edited by Gennaro AR, 19th ed. Pennsylvania: Mack Publishing Company, 1995), and includes but is not limited to: pH regulator, surfactant, adjuvant, ionic strength enhancer. For example, the pH regulator includes but is not limited to phosphate buffer, the surfactant includes but is not limited to cationic, anionic or non-ionic surfactant, such as Tween-80; the ionic strength enhancer includes bit is not limited to sodium chloride.

The term “adjuvant” refers to a nonspecific immunopotentiator that, when delivered to the body together with an antigen or in advance, can enhance the body's immune response to the antigen or change the type of immune response. There are many types of adjuvants, including but not limited to aluminum adjuvants (e.g., aluminum hydroxide), Freund's adjuvants (e.g., complete Freund's adjuvant and incomplete Freund's adjuvant). Corynebacterium brevis, lipopolysaccharide, cytokine, etc. Freund's adjuvant is currently the most commonly used adjuvant in animal experiments. Aluminum hydroxide adjuvant is more commonly used in clinical trials.

Another aspect of the present invention relates to a use of the isolated mRNA molecule, the isolated DNA molecule, the recombinant plasmid or the recombinant host cell of any one of the items of the present invention in the manufacture of a medicament for preventing or treating variola or monkeypox virus infection; preferably, the medicament is a vaccine.

The isolated mRNA molecule, the isolated DNA molecule, the recombinant plasmid or the recombinant host cell of any one of the items of the present invention are used to prevent or treat variola or monkeypox virus infection.

Another aspect of the preset invention relates to a method for preventing or treating variola or monkeypox virus infection, comprising a step of administering to a subject in need thereof effective amount of the isolated mRNA molecule, the isolated DNA molecule, the recombinant plasmid or the recombinant host cell of ay one of the items of the present invention.

The term “subject” may refer to a patient or other animal, especially a mammal, such as human, dog, monkey, cattle, horse, that receives the medicament (e.g., vaccine preparation) or active pharmaceutical ingredient (e.g., the isolated mRNA molecule, the isolated DNA molecule, the recombinant plasmid or the recombinant host cell of any one of the items of the present invention) of the present invention to treat, prevent, alleviate and/or relieve the disease or condition described in the present invention.

The term “effective amount” refers to an amount sufficient to obtain or at least partially obtain the desired effect. For example, an effective amount for preventing a disease (e.g., variola or monkeypox virus infection) refers to an amount sufficient to prevent, arrest, or delay the occurrence of a disease (e.g., variola or monkeypox virus infection); an effective amount for treating a disease (e.g., variola or monkeypox virus infection) refers to an amount sufficient to cure or at least partially prevent the disease and its complications in a patient who already has the disease. Determining such an effective amount is well within the capabilities of those skilled in the art. For example, an effective amount for therapeutic use will depend on the severity of the disease to be treated, the overall state of the patient's own immune system, the patient's general condition such as age, weight end sex, the mode of administration of the medicament, and other treatments administered simultaneously, etc.

However, it should be recognized that the total daily dose of the medicament (e.g., vaccine preparation) or the active ingredient of the medicament (e.g., the isolated mRNA molecule described in any one of the items of the present invention, the isolated DNA molecule described in any one of the items of the present invention, the recombinant plasmid of the present invention, or the recombinant host cell of the present invention) of the present invention must be determined by the attending physician within the scope of reliable medical judgment. For any particular patient, the specific therapeutically effective dosage level must be determined based on a variety of factors, including the severity of the disease to be treated (e.g., variola or monkeypox virus infection); the activity of the specific vaccine formulation used; the patient's age, weight, general health, sex and diet administration time, route of administration ad excretion rate; duration of treatment; other drugs used at the same time; and similar factors known in the medical field. For example, it is practiced in the art to start the administration of a dose lower then the level required to obtain the desired therapeutic effect and gradually increase the dose until the desired effect is achieved.

The mRNA vaccine of the present invention can be prepared by referring to the following steps:
(1) Gene cloning: the target sequence (e.g., SEQ ID NO: 5) is cloned into a plasmid vector, a monoclone is selected and sequenced to obtain a positive done containing the target gene, and the positive done strain is subjected to fermentation and extraction to obtain a circular plasmid template;
(2) Preparation of in vitro transcription (IVT) template: the circular plasmid is enzymatically linearized by restriction endonuclease (e.g., BspQI) and the linearized plasmid is purified and used as a starting template for mRNA synthesis;
(3) Synthesis and purification of mRNA: the target mRNA is synthesized by in vitro transcription reaction IVT using the linearized plasmid as a template, and purified to obtain a high-purity mRNA molecule;
(4) Preparation and purification of LNP-mRNA lipid nanoparticle complex: the purified mRNA stock solution and four LNPs (lipid nanoparticles) are mixed by a microfluidic device to form an mRNA-LNP complex, the mRNA-LNP complex is subjected to dilution and medium exchange with PBS and concentrated by ultrafiltration to obtain an mRNA vaccine preparation.

### Beneficial Effects of the Invention

The present invention has achieved one or more of the following technical effects (1) to (4):
(1) The mRNA vaccine of the present invention can induce high levels of specific binding antibodies against each of the VACV antigens (A27, A33, L1 and B5) In vivo, and the levels of antibodies against the four antigens are significantly improved after the second immunization.
(2) The mRNA vaccine of the present invention can induce high levels of cross-binding antibodies against each of the MPXV antigens (A29, A35, M1 and B6) in vivo, and the antibody levels are significantly improved after the second immunization.
(3) The mRNA vaccine of the present invention can induce significant VACV neutralizing antibodies, and the final neutralizing antibody titer is greater than 1000.
(4) The mRNA vaccine of the present invention can induce specific cellular immune responses.

### BRIEF DESCRIPTION OF THE DRAWINGS

FIG. 1 shows the ABLB-pUCYH plasmid restriction electrophoresis, wherein, M denotes markers, I denotes plasmids before linearization, and II denotes plasmids after linearization.

FIG. 2A shows the Gel electrophoresis of the purified mRNA.

FIG. 2B shows the HPLC-SEC analysis of the purified mRNA.

FIG. 3 shows the particle size detection curves of ALAB mRNA-LNP vaccine preparations.

FIG. 4A shows the detection curve of VACV A27 antigen-specific IgG titer in immunized mouse serum.

FIG. 4B shows the detection curve of VACV L1 antigen-specific IgG titer in immunized mouse serum.

FIG. 4C shows the detection curve of VACV A33 antigen-specific IgG titer in immunized mouse serum.

FIG. 4D shows the detection curve of VACV B5 antigen-specific IgG titer in immunized mouse serum.

FIG. 5A shows the detection curve of MPXV A29 antigen cross-reactive IgG titer in immunized mouse serum.

FIG. 5B shows the detection curve of MPXV M1 antigen cross-reactive IgG titer in immunized mouse serum.

FIG. 5C shows the detection curve of MPXV A35 antigen cross-reactive IgG titer in immunized mouse serum.

FIG. 5D shows the detection curve of MPXV B6 antigen cross-reactive IgG titer in immunized mouse serum.

FIG. 6A shows the representative results of VACV neutralization activity of mouse sera at different dilutions in the empty group (Empty) and the vaccine group (ALAB) 14 days after the second immunization.

FIG. 6B shows the summary data of VACV neutralization activity of mouse sera in the empty group (Empty) and the vaccine group (ALAB) 14 days after the second immunization.

FIG. 7A shows the representative results of ELISPOT of mouse antigen-specific IFN-γ positive cell immune response 35 days after the second immunization.

FIG. 7B shows the summary data of ELISPOT of mouse antigen-specific IFN-γ positive cell immune response 35 days after the second immunization.

Several sequences involved in the present invention are as follows.

1. mRNA full-length nucleic acid sequence:
(SEQ ID NO: 1)
GAGACUCUUCUGGUCCCCACAGACUCAGAGAGAACCCGCCACCAUGGACG
GAACUCUGUUCCCCGGCGACGACGAUCUGGCCAUCCCCGCCACCGAGUUU
UUCAGCACAAAGGCCGCUAAGAAGCCUGAAGCCAAGAGAGAGGCCAUCGU
GAAGGCCGAUGAAGAUGAUAACGAGGAAACCCUGAAGCAGAGACUGACAA
AUCUCGAAAAGAAGAUCACCAACGUGACCACAAAGUUCGAGCAAAUUGAA
AAGUGCUGCAAGCGGAACGAUGAAGUUCUAUUCCGGCUGGAAAACCACGC
CGAGACCCUGAGAGCCGCCAUGAUCAGCCUGGCCAAGAAAAUCGACGUGC
AAACAGGCAGACGGCCUUACGAAAGAAGAAAGAGGGGCAGCGGCGCCACA
AACUUCUCACUGCUGAAACAGGCCGGCGACGUGGAGGAAAAUCCUGGCCC
UAUGGGCGCCGCCGCUAGCAUCCAGACAACAGUGAACACCCUGAGCGAGC
GGAUCAGCAGCAAGCUGGAGCAGGAGGCCAAUGCCUCUGCCCAGACCAAA
UGCGACAUCGAGAUCGGCAAUUUUUACAUCAGACAGAACCACGGGUGCAA
CCUGACCGUGAAGAACAUGUGCUCCGCUGACGCGGAUGCCCAGCUGGAUG
CCGUCCUGUCUGCCGCCACUGAAACCUACAGCGGCCUGACACCAGAACAA
AAGGCCUACGUUCCUGCCAUGUUCACCGCCGCCCUGAACAUCCAGACCUC
UGUGAACACCGUGGUGCGGGACUUCGAGAAUUACGUGAAGCAGACAUGCA
ACAGCAGCGCUGUGGUGGACAACAAGCUGAAAAUUCAGAACGUGAUCAUA
GACGAGUGUUACGGAGCCCCUGGCAGCCCUACCAAUCUCGAGUUCAUCAA
CACCGGCAGCAGCAAGGGCAAUUGCGCCAUCAAGGCCCUGAUGCAGCUGA
CCACGAAGGCUACCACCCAGAUCGCCCCUAAGCAGGUGGCCGGCACCGGC
GUGCAGUUCUACAUGAUCGUGAUCGGCGUGAUCAUUCUGGCCGCCCUCUU
UAUGUACUACGCCAAGCGUAUGCUGUUCACCAGCACUAAUGAUAAGAUCA
AGCUGAUUCUGGCUAAUAAAGAGAACGUGCACUGGACCACAUAUAUGGAC
ACCUUCUUUAGAACCAGCCCCAUGGUGAUCGCCACCACCGACAUGCAAAA
UAGAAGAAAGAGGGGCUCUGGAGCCACCAACUUUAGCCUGCUGAAGCAGG
CUGGAGAUGUGGAAGAGAACCCUGGCCCUAUGAUGACACCUGAGAACGAU
GAGGAGCAAACCUCUGUGUUCAGCGCCACGGUGUACGGCGAUAAGAUCCA
GGGUAAGAAUAAGCGGAAGAGAGUGAUCGGCCUGUGUAUCCGGAUCUCUA
UGGUGAUCAGCUUGCUGAGCAUGAUCACCAUGUCUGCCUUCCUGAUCGUG
CGGCUGAACCAGUGCAUGUCCGCCAACGAAGCCGCCAUCACCGACGCCGC
AGUGGCCGUGGCCGCAGCUAGCAGCACCCAUAGAAAAGUGGCAAGCAGCA
CAACCCAGUACGAUCACAAAGAGUCCUGUAACGGCCUGUACUACCAAGGC
UCUUGCUACAUCCUGCACAGCGACUAUCAGCUGUUCUCAGACGCCAAGGC
UAACUGCACUGCCGAAUCCAGCACCCUGCCUAACAAAAGCGACGUGCUGA
UUACCUGGCUGAUCGACUACGUGGAGGACACCUGGGGCUCUGAUGGCAAC
CCCAUCACUAAAACCACCAGCGAUUACCAGGACAGCGACGUCAGCCAGGA
GGUCAGAAAAUAUUUCUGCGUGAAGACCAUGAACCGGAGAAAGAGAGGCA
GCGGCGCCACCAACUUCUCACUGCUGAAGCAGGCCGGAGAUGUGGAAGAA
AACCCAGGCCCAAUGAAGACCAUCAGCGUGGUGACACUGCUGUGCGUGCU
CCCCGCUGUUGUGUACUCCACCUGUACAGUGCCUACAAUGAACAACGCCA
AGCUGACAAGCACCGAGACCAGCUUCAACGAUAAGCAGAAGGUGACCUUC
ACCUGUGACCAAGGAUACCACAGCCUGGACCCUAACGCUGUGUGCGAGAC
AGACAAGUGGAAGUACGAGAACCCUUGUAAAAAGAUGUGUACAGUGUCUG
ACUACGUGAGCGAGCUGUACGACAAGCCCCUGUACGAGGUCAAUUCCACC
AUGACCCUGAGCUGUAACGGCGAGACAAAGUACUUCAGAUGCGAGGAAAA
GAACGGCAACACAAGCUGGAACGACACCGUGACUUGCCCCAACGCCGAAU
GCCAGCCGCUGCAGCUGGAGCACGGCAGCUGCCAGCCUGUCAAAGAGAAG
UAUAGCUUCGGUGAAUACAUUACAAUCAAUUGCGACGUGGGCUACGAGGU
GAUCGGCGCCAGCUACAUCAGCUGUACCGCUAAUAGCUGGAACGUCAUCC
CUUCCUGCCAGCAGAAGUGCGACAUGCCUUCUCUGUCUAACGGCCUGAUC
UCCGGAUCAACAUUUUCCAUCGGCGGCGUGAUCCACCUGAGCUGCAAGUC
CGGCUUCAUCCUGACCGGCUCCCCUUCUAGCACCUGCAUCGAUGGCAAGU
GGAACCCCAUCCUUCCAACCUGCGUGAGAUCCAACAAGGAGUUCGACCCC
GUGGACGACGGCCCCGACGACGAGACAGACCUCAGCAAGCUGAGCAAGGA
UGUGGUCCAGUACGAGCAGGAAAUCGAGAGCCUAGAGGCCACCUACCACA
UCAUCAUCGUGGCUCUGACAAUCAUGGGAGUGAUCUUCCUGAUCUCUGUU
AUCGUGCUGGUGUGCUCCUGUGACAAAAACAACGACCAGUACAAGUUCCA
CAAGCUGCUGCCUUGAUAAGCUGGAGCCUCGGUGGCCAUGCUUCUUGCCC
CUUGGGCCUCCCCCCAGCCCCUCCUCCCCUUCCUGCACCCGUACCCCCGU
GGUCUUUGAAUAAAGUCUGAGUGGGCGAAAAAAAAAAAAAAAAAAAAAAA
AAAAAAAAAAAAAAAAAAAAAAAAAAAAAAAAAAAAAGAAAAAAAAAAAA
AAAAAAAAAAAAAAAAAAAAAAAAAAAAAAAAAAAAAAAAAAAAAAAA
2. Protein sequence encoded by CDS:
(SEQ ID NO: 2)
MDGTLFPGDDDLAIPATEFFSTKAAKKPEAKREAIVKADEDDNEETLKQR
LTNLEKKITNVTTKFEQIEKCCKRNDEVLFRLENHAETLRAAMISLAKKI
DVQTGRRPYERRKRGSGATNFSLLKQAGDVEENPGPMGAAASIQTTVNTL
SERISSKLEQEANASAQTKCDIEIGNFYIRQNHGCNLTVKNMCSADADAQ
LDAVLSAATETYSGLTPEQKAYVPAMFTAALNIQTSVNTVVRDFENYVKQ
TCNSSAVVDNKLKIQNVIIDECYGAPGSPTNLEFINTGSSKGNCAIKALM
QLTTKATTQIAPKQVAGTGVQFYMIVIGVIILAALFMYYAKRMLFTSTND
KIKLILANKENVHWTTYMDTFFRTSPMVIATTDMQNRRKRGSGATNFSLL
KQAGDVEENPGPMMTPENDEEQTSVFSATVYGDKIQGKNKRKRVIGLCIR
ISMVISLLSMITMSAFLIVRLNQCMSANEAAITDAAVAVAAASSTHRKVA
SSTTQYDHKESCNGLYYQGSCYILHSDYQLFSDAKANCTAESSTLPNKSD
VLITWLIDYVEDTWGSDGNPITKTTSDYQDSDVSQEVRKYFCVKTMNRRK
RGSGATNFSLLKQAGDVEENPGPMKTISVVTLLCVLPAVVYSTCTVPTMN
NAKLTSTETSFNDKQKVTFTCDQGYHSLDPNAVCETDKWKYENPCKKMCT
VSDYVSELYDKPLYEVNSTMTLSCNGETKYFRCEEKNGNTSWNDTVTCPN
AECQPLQLEHGSCQPVKEKYSFGEYITINCDVGYEVIGASYISCTANSWN
VIPSCQQKCDMPSLSNGLISGSTFSIGGVIHLSCKSGFILTGSPSSTCID
GKWNPILPTCVRSNKEFDPVDDGPDDETDLSKLSKDVVQYEQEIESLEAT
YHIIIVALTIMGVIFLISVIVLVCSCDKNNDQYKFHKLLP
3. Coding region (DNA nucleic acid sequence)
for expressing four proteins A27, L1, A33
and BS in tandem:
(SEQ ID NO: 3)
ATGGACGGAACTCTTTTCCCCGGAGATGACGATCTTGCAATTCCAGCAAC
TGAATTTTTTTCTACAAAGGCTGCTAAAAAGCCAGAGGCTAAACGCGAAG
CAATTGTTAAAGCCGATGAAGACGACAATGAGGAAACTCTCAAACAACGG
CTAACTAATTTGGAAAAAAAGATTACTAATGTAACAACAAAGTTTGAACA
AATAGAAAAGTGTTGTAAACGCAACGATGAAGTTCTATTTAGGTTGGAAA
ATCACGCTGAAACTCTAAGAGCGGCTATGATATCTCTGGCTAAAAAGATT
GATGTTCAGACTGGACGGCGTCCATATGAGCGCAGAAAGAGAGGCTCCGG
CGCCACAAACTTCAGCCTGCTGAAGCAGGCTGGCGACGTCGAGGAAAACC
CCGGCCCTATGGGTGCCGCAGCAAGCATACAGACGACGGTGAATACACTC
AGCGAACGTATCTCGTCTAAATTAGAACAAGAAGCGAACGCTAGTGCTCA
AACAAAATGTGATATAGAAATCGGAAATTTTTATATCCGACAAAACCATG
GATGTAACCTCACTGTTAAAAATATGTGCTCTGCGGACGCGGATGCTCAG
TTGGATGCTGTGTTATCAGCCGCTACAGAAACATATAGTGGATTAACACC
GGAACAAAAAGCATACGTACCAGCTATGTTTACTGCTGCGTTAAACATTC
AGACAAGTGTAAACACTGTTGTTAGAGATTTTGAAAATTATGTGAAACAA
ACTTGTAATTCTAGCGCGGTCGTCGATAACAAATTAAAGATACAAAACGT
AATCATAGATGAATGTTACGGAGCCCCAGGATCTCCAACAAATTTGGAAT
TTATTAATACAGGATCTAGCAAAGGAAATTGTGCCATTAAGGCGTTGATG
CAATTGACTACTAAGGCCACTACTCAAATAGCACCTAAACAAGTTGCTGG
TACAGGAGTTCAGTTTTATATGATTGTTATCGGTGTTATAATATTGGCAG
CGTTGTTTATGTACTATGCCAAGCGTATGCTGTTCACATCCACCAATGAT
AAAATCAAACTTATTTTAGCCAATAAGGAAAACGTCCATTGGACTACTTA
CATGGACACATTCTTTAGAACTTCTCCGATGGTTATTGCTACCACGGATA
TGCAAAACCGCAGAAAGAGAGGCTCCGGCGCCACAAACTTCAGCCTGCTG
AAGCAGGCTGGCGACGTCGAGGAAAACCCCGGCCCTATGATGACACCAGA
AAACGACGAAGAGCAGACATCTGTGTTCTCCGCTACTGTTTACGGAGACA
AAATTCAGGGAAAGAATAAACGCAAACGCGTGATTGGTCTATGTATTAGA
ATATCTATGGTTATTTCACTACTATCTATGATTACCATGTCCGCGTTTCT
CATAGTGCGCCTAAATCAATGCATGTCTGCTAACGAGGCTGCTATTACTG
ACGCCGCTGTTGCCGTTGCTGCTGCATCATCTACTCATAGAAAGGTTGCG
TCTAGCACTACACAATATGATCACAAAGAAAGCTGTAATGGTTTATATTA
CCAGGGTTCTTGTTATATATTACATTCAGACTACCAGTTATTCTCGGATG
CTAAAGCAAATTGCACTGCGGAATCATCAACACTACCCAATAAATCCGAT
GTCTTGATTACCTGGCTCATTGATTATGTTGAGGATACATGGGGATCTGA
TGGTAATCCAATTACAAAAACTACATCCGATTATCAAGATTCTGATGTAT
CACAAGAAGTTAGAAAGTATTTTTGTGTTAAAACAATGAACCGCAGAAAG
AGAGGCTCCGGCGCCACAAACTTCAGCCTGCTGAAGCAGGCTGGCGACGT
CGAGGAAAACCCCGGCCCTATGAAAACGATTTCCGTTGTTACGTTGTTAT
GCGTACTACCTGCTGTTGTTTATTCAACATGTACTGTACCCACTATGAAT
AACGCTAAATTAACGTCTACCGAAACATCGTTTAATGATAAACAGAAAGT
TACATTTACATGTGATCAGGGATATCATTCTTTGGATCCAAATGCTGTCT
GCGAAACAGATAAATGGAAATACGAAAATCCATGCAAGAAAATGTGCACA
GTTTCTGATTATGTCTCTGAATTATATGATAAGCCATTATACGAAGTGAA
TTCCACCATGACACTAAGTTGCAACGGCGAAACAAAATATTTTCGTTGCG
AAGAAAAAAATGGAAATACTTCTTGGAATGATACTGTTACGTGTCCTAAT
GCGGAATGTCAACCTCTTCAATTAGAACACGGATCGTGTCAACCAGTTAA
AGAAAAATACTCATTTGGGGAATATATAACTATCAACTGTGATGTTGGAT
ATGAGGTTATTGGTGCTTCGTACATAAGTTGTACAGCTAATTCTTGGAAT
GTTATTCCATCATGTCAACAAAAATGTGATATGCCGTCTCTATCTAACGG
ATTAATTTCCGGATCTACATTTTCTATCGGTGGCGTTATACATCTTAGTT
GTAAAAGTGGTTTTATACTAACGGGATCTCCATCATCCACATGTATCGAC
GGTAAATGGAATCCCATACTCCCAACATGTGTACGATCTAACAAAGAATT
TGATCCAGTGGATGATGGTCCCGACGATGAGACAGATTTGAGCAAACTCT
CGAAAGACGTTGTACAATATGAACAAGAAATAGAATCGTTAGAAGCAACT
TATCATATAATCATAGTGGCGTTAACAATTATGGGCGTCATATTTTTAAT
CTCCGTTATAGTATTAGTTTGTTCCTGTGACAAAAATAATGACCAATATA
AGTTCCATAAATTGCTACCG
4. Coding region (DNA nucleic acid sequence)
optimized for expressing four proteins A27,
L1, A33 and B5 in tandem:
(SEQ ID NO: 4)
ATGGACGGAACTCTGTTCCCCGGCGACGACGATCTGGCCATCCCCGCCAC
CGAGTTTTTCAGCACAAAGGCCGCTAAGAAGCCTGAAGCCAAGAGAGAGG
CCATCGTGAAGGCCGATGAAGATGATAACGAGGAAACCCTGAAGCAGAGA
CTGACAAATCTCGAAAAGAAGATCACCAACGTGACCACAAAGTTCGAGCA
AATTGAAAAGTGCTGCAAGCGGAACGATGAAGTTCTATTCCGGCTGGAAA
ACCACGCCGAGACCCTGAGAGCCGCCATGATCAGCCTGGCCAAGAAAATC
GACGTGCAAACAGGCAGACGGCCTTACGAAAGAAGAAAGAGGGGCAGCGG
CGCCACAAACTTCTCACTGCTGAAACAGGCCGGCGACGTGGAGGAAAATC
CTGGCCCTATGGGCGCCGCCGCTAGCATCCAGACAACAGTGAACACCCTG
AGCGAGCGGATCAGCAGCAAGCTGGAGCAGGAGGCCAATGCCTCTGCCCA
GACCAAATGCGACATCGAGATCGGCAATTTTTACATCAGACAGAACCACG
GGTGCAACCTGACCGTGAAGAACATGTGCTCCGCTGACGCGGATGCCCAG
CTGGATGCCGTCCTGTCTGCCGCCACTGAAACCTACAGCGGCCTGACACC
AGAACAAAAGGCCTACGTTCCTGCCATGTTCACCGCCGCCCTGAACATCC
AGACCTCTGTGAACACCGTGGTGCGGGACTTCGAGAATTACGTGAAGCAG
ACATGCAACAGCAGCGCTGTGGTGGACAACAAGCTGAAAATTCAGAACGT
GATCATAGACGAGTGTTACGGAGCCCCTGGCAGCCCTACCAATCTCGAGT
TCATCAACACCGGCAGCAGCAAGGGCAATTGCGCCATCAAGGCCCTGATG
CAGCTGACCACGAAGGCTACCACCCAGATCGCCCCTAAGCAGGTGGCCGG
CACCGGCGTGCAGTTCTACATGATCGTGATCGGCGTGATCATTCTGGCCG
CCCTCTTTATGTACTACGCCAAGCGTATGCTGTTCACCAGCACTAATGAT
AAGATCAAGCTGATTCTGGCTAATAAAGAGAACGTGCACTGGACCACATA
TATGGACACCTTCTTTAGAACCAGCCCCATGGTGATCGCCACCACCGACA
TGCAAAATAGAAGAAAGAGGGGCTCTGGAGCCACCAACTTTAGCCTGCTG
AAGCAGGCTGGAGATGTGGAAGAGAACCCTGGCCCTATGATGACACCTGA
GAACGATGAGGAGCAAACCTCTGTGTTCAGCGCCACGGTGTACGGCGATA
AGATCCAGGGTAAGAATAAGCGGAAGAGAGTGATCGGCCTGTGTATCCGG
ATCTCTATGGTGATCAGCTTGCTGAGCATGATCACCATGTCTGCCTTCCT
GATCGTGCGGCTGAACCAGTGCATGTCCGCCAACGAAGCCGCCATCACCG
ACGCCGCAGTGGCCGTGGCCGCAGCTAGCAGCACCCATAGAAAAGTGGCA
AGCAGCACAACCCAGTACGATCACAAAGAGTCCTGTAACGGCCTGTACTA
CCAAGGCTCTTGCTACATCCTGCACAGCGACTATCAGCTGTTCTCAGACG
CCAAGGCTAACTGCACTGCCGAATCCAGCACCCTGCCTAACAAAAGCGAC
GTGCTGATTACCTGGCTGATCGACTACGTGGAGGACACCTGGGGCTCTGA
TGGCAACCCCATCACTAAAACCACCAGCGATTACCAGGACAGCGACGTCA
GCCAGGAGGTCAGAAAATATTTCTGCGTGAAGACCATGAACCGGAGAAAG
AGAGGCAGCGGCGCCACCAACTTCTCACTGCTGAAGCAGGCCGGAGATGT
GGAAGAAAACCCAGGCCCAATGAAGACCATCAGCGTGGTGACACTGCTGT
GCGTGCTCCCCGCTGTTGTGTACTCCACCTGTACAGTGCCTACAATGAAC
AACGCCAAGCTGACAAGCACCGAGACCAGCTTCAACGATAAGCAGAAGGT
GACCTTCACCTGTGACCAAGGATACCACAGCCTGGACCCTAACGCTGTGT
GCGAGACAGACAAGTGGAAGTACGAGAACCCTTGTAAAAAGATGTGTACA
GTGTCTGACTACGTGAGCGAGCTGTACGACAAGCCCCTGTACGAGGTCAA
TTCCACCATGACCCTGAGCTGTAACGGCGAGACAAAGTACTTCAGATGCG
AGGAAAAGAACGGCAACACAAGCTGGAACGACACCGTGACTTGCCCCAAC
GCCGAATGCCAGCCGCTGCAGCTGGAGCACGGCAGCTGCCAGCCTGTCAA
AGAGAAGTATAGCTTCGGTGAATACATTACAATCAATTGCGACGTGGGCT
ACGAGGTGATCGGCGCCAGCTACATCAGCTGTACCGCTAATAGCTGGAAC
GTCATCCCTTCCTGCCAGCAGAAGTGCGACATGCCTTCTCTGTCTAACGG
CCTGATCTCCGGATCAACATTTTCCATCGGCGGCGTGATCCACCTGAGCT
GCAAGTCCGGCTTCATCCTGACCGGCTCCCCTTCTAGCACCTGCATCGAT
GGCAAGTGGAACCCCATCCTTCCAACCTGCGTGAGATCCAACAAGGAGTT
CGACCCCGTGGACGACGGCCCCGACGACGAGACAGACCTCAGCAAGCTGA
GCAAGGATGTGGTCCAGTACGAGCAGGAAATCGAGAGCCTAGAGGCCACC
TACCACATCATCATCGTGGCTCTGACAATCATGGGAGTGATCTTCCTGAT
CTCTGTTATCGTGCTGGTGTGCTCCTGTGACAAAAACAACGACCAGTACA
AGTTCCACAAGCTGCTGCCT
5. Cloned fragment (DNA nucleic acid
sequence):
(SEQ ID NO: 5)
TAATACGACTCACTATAAGACTCTTCTGGTCCCCACAGACTCAGAGAGAA
CCCGCCACCATGGACGGAACTCTGTTCCCCGGCGACGACGATCTGGCCAT
CCCCGCCACCGAGTTTTTCAGCACAAAGGCCGCTAAGAAGCCTGAAGCCA
AGAGAGAGGCCATCGTGAAGGCCGATGAAGATGATAACGAGGAAACCCTG
AAGCAGAGACTGACAAATCTCGAAAAGAAGATCACCAACGTGACCACAAA
GTTCGAGCAAATTGAAAAGTGCTGCAAGCGGAACGATGAAGTTCTATTCC
GGCTGGAAAACCACGCCGAGACCCTGAGAGCCGCCATGATCAGCCTGGCC
AAGAAAATCGACGTGCAAACAGGCAGACGGCCTTACGAAAGAAGAAAGAG
GGGCAGCGGCGCCACAAACTTCTCACTGCTGAAACAGGCCGGCGACGTGG
AGGAAAATCCTGGCCCTATGGGCGCCGCCGCTAGCATCCAGACAACAGTG
AACACCCTGAGCGAGOGGATCAGCAGCAAGCTGGAGCAGGAGGCCAATGC
CTCTGCCCAGACCAAATGCGACATCGAGATCGGCAATTTTTACATCAGAC
AGAACCACGGGTGCAACCTGACCGTGAAGAACATGTGCTCCGCTGACGCG
GATGCCCAGCTGGATGCCGTCCTGTCTGCCGCCACTGAAACCTACAGCGG
CCTGACACCAGAACAAAAGGCCTACGTTCCTGCCATGTTCACCGCCGCCC
TGAACATCCAGACCTCTGTGAACACCGTGGTGCGGGACTTCGAGAATTAC
GTGAAGCAGACATGCAACAGCAGCGCTGTGGTGGACAACAAGCTGAAAAT
TCAGAACGTGATCATAGACGAGTGTTACGGAGCCCCTGGCAGCCCTACCA
ATCTCGAGTTCATCAACACCGGCAGCAGCAAGGGCAATTGCGCCATCAAG
GCCCTGATGCAGCTGACCACGAAGGCTACCACCCAGATCGCCCCTAAGCA
GGTGGCCGGCACCGGCGTGCAGTTCTACATGATCGTGATCGGCGTGATCA
TTCTGGCCGCCCTCTTTATGTACTACGCCAAGCGTATGCTGTTCACCAGC
ACTAATGATAAGATCAAGCTGATTCTGGCTAATAAAGAGAACGTGCACTG
GACCACATATATGGACACCTTCTTTAGAACCAGCCCCATGGTGATCGCCA
CCACCGACATGCAAAATAGAAGAAAGAGGGGCTCTGGAGCCACCAACTTT
AGCCTGCTGAAGCAGGCTGGAGATGTGGAAGAGAACCCTGGCCCTATGAT
GACACCTGAGAACGATGAGGAGCAAACCTCTGTGTTCAGCGCCACGGTGT
ACGGCGATAAGATCCAGGGTAAGAATAAGCGGAAGAGAGTGATCGGCCTG
TGTATCCGGATCTCTATGGTGATCAGCTTGCTGAGCATGATCACCATGTC
TGCCTTCCTGATCGTGCGGCTGAACCAGTGCATGTCCGCCAACGAAGCCG
CCATCACCGACGCCGCAGTGGCCGTGGCCGCAGCTAGCAGCACCCATAGA
AAAGTGGCAAGCAGCACAACCCAGTACGATCACAAAGAGTCCTGTAACGG
CCTGTACTACCAAGGCTCTTGCTACATCCTGCACAGCGACTATCAGCTGT
TCTCAGACGCCAAGGCTAACTGCACTGCCGAATCCAGCACCCTGCCTAAC
AAAAGCGACGTGCTGATTACCTGGCTGATCGACTACGTGGAGGACACCTG
GGGCTCTGATGGCAACCCCATCACTAAAACCACCAGCGATTACCAGGACA
GCGACGTCAGCCAGGAGGTCAGAAAATATTTCTGCGTGAAGACCATGAAC
CGGAGAAAGAGAGGCAGCGGCGCCACCAACTTCTCACTGCTGAAGCAGGC
CGGAGATGTGGAAGAAAACCCAGGCCCAATGAAGACCATCAGCGTGGTGA
CACTGCTGTGCGTGCTCCCCGCTGTTGTGTACTCCACCTGTACAGTGCCT
ACAATGAACAACGCCAAGCTGACAAGCACCGAGACCAGCTTCAACGATAA
GCAGAAGGTGACCTTCACCTGTGACCAAGGATACCACAGCCTGGACCCTA
ACGCTGTGTGCGAGACAGACAAGTGGAAGTACGAGAACCCTTGTAAAAAG
ATGTGTACAGTGTCTGACTACGTGAGCGAGCTGTACGACAAGCCCCTGTA
CGAGGTCAATTCCACCATGACCCTGAGCTGTAACGGCGAGACAAAGTACT
TCAGATGCGAGGAAAAGAACGGCAACACAAGCTGGAACGACACCGTGACT
TGCCCCAACGCCGAATGCCAGCCGCTGCAGCTGGAGCACGGCAGCTGCCA
GCCTGTCAAAGAGAAGTATAGCTTCGGTGAATACATTACAATCAATTGCG
ACGTGGGCTACGAGGTGATCGGCGCCAGCTACATCAGCTGTACCGCTAAT
AGCTGGAACGTCATCCCTTCCTGCCAGCAGAAGTGCGACATGCCTTCTCT
GTCTAACGGCCTGATCTCCGGATCAACATTTTCCATCGGCGGCGTGATCC
ACCTGAGCTGCAAGTCCGGCTTCATCCTGACCGGCTCCCCTTCTAGCACC
TGCATCGATGGCAAGTGGAACCCCATCCTTCCAACCTGCGTGAGATCCAA
CAAGGAGTTCGACCCCGTGGACGACGGCCCCGACGACGAGACAGACCTCA
GCAAGCTGAGCAAGGATGTGGTCCAGTACGAGCAGGAAATCGAGAGCCTA
GAGGCCACCTACCACATCATCATCGTGGCTCTGACAATCATGGGAGTGAT
CTTCCTGATCTCTGTTATCGTGCTGGTGTGCTCCTGTGACAAAAACAACG
ACCAGTACAAGTTCCACAAGCTGCTGCCTTGATAAGCTGGAGCCTCGGTG
GCCATGCTTCTTGCCCCTTGGGCCTCCCCCCAGCCCCTCCTCCCCTTCCT
GCACCCGTACCCCCGTGGTCTTTGAATAAAGTCTGAGTGGGCGGCAAAAA
AAAAAAAAAAAAAAAAAAAAAAAAAAAAAAAAAAAAAAAAAAAAAAAAAA
AAAAAACGAAAAAAAAAAAAAAAAAAAAAAAAAAAAAAAAAAAAAAAAAA
AAAAAAAAAAAAAAAAAAAGAAGAGC
6. A27 Protein
(SEQ ID NO: 6)
MDGTLFPGDDDLAIPATEFFSTKAAKKPEAKREAIVKADEDDNEETLKQR
LTNLEKKITNVTTKFEQIEKCCKRNDEVLFRLENHAETLRAAMISLAKKI
DVQTGRRPYE
7. L1 protein
(SEQ ID NO: 7)
MGAAASIQTTVNTLSERISSKLEQEANASAQTKCDIEIGNFYIRQNHGCN
LTVKNMCSADADAQLDAVLSAATETYSGLTPEQKAYVPAMFTAALNIQTS
VNTVVRDFENYVKQTCNSSAVVDNKLKIQNVIIDECYGAPGSPTNLEFIN
TGSSKGNCAIKALMQLTTKATTQIAPKQVAGTGVQFYMIVIGVIILAALF
MYYAKRMLFTSTNDKIKLILANKENVHWTTYMDTFFRTSPMVIATTDMQN
8. A33 protein
(SEQ ID NO: 8)
MMTPENDEEQTSVFSATVYGDKIQGKNKRKRVIGLCIRISMVISLLSMIT
MSAFLIVRLNQCMSANEAAITDAAVAVAAASSTHRKVASSTTQYDHKESC
NGLYYQGSCYILHSDYQLFSDAKANCTAESSTLPNKSDVLITWLIDYVED
TWGSDGNPITKTTSDYQDSDVSQEVRKYFCVKTMN
9. B5 protein
(SEQ ID NO: 9)
MKTISVVTLLCVLPAVVYSTCTVPTMNNAKLTSTETSFNDKQKVTFTCDQ
GYHSLDPNAVCETDKWKYENPCKKMCTVSDYVSELYDKPLYEVNSTMTLS
CNGETKYFRCEEKNGNTSWNDTVTCPNAECQPLQLEHGSCQPVKEKYSFG
EYITINCDVGYEVIGASYISCTANSWNVIPSCQQKCDMPSLSNGLISGST
FSIGGVIHLSCKSGFILTGSPSSTCIDGKWNPILPTCVRSNKEFDPVDDG
PDDETDLSKLSKDVVQYEQEIESLEATYHIIIVALTIMGVIFLISVIVLV
CSCDKNNDQYKFHKLLP
10. Amino acid sequence of linker
(SEQ ID NO: 10)
RRKRGSGATNFSLLKQAGDVEENPGP
11. Coding region in mRNA molecule
(SEQ ID NO: 11)
AUGGACGGAACUCUGUUCCCCGGCGACGACGAUCUGGCCAUCCCCGCCAC
CGAGUUUUUCAGCACAAAGGCCGCUAAGAAGCCUGAAGCCAAGAGAGAGG
CCAUCGUGAAGGCCGAUGAAGAUGAUAACGAGGAAACCCUGAAGCAGAGA
CUGACAAAUCUCGAAAAGAAGAUCACCAACGUGACCACAAAGUUCGAGCA
AAUUGAAAAGUGCUGCAAGCGGAACGAUGAAGUUCUAUUCCGGCUGGAAA
ACCACGCCGAGACCCUGAGAGCCGCCAUGAUCAGCCUGGCCAAGAAAAUC
GACGUGCAAACAGGCAGACGGCCUUACGAAAGAAGAAAGAGGGGCAGCGG
CGCCACAAACUUCUCACUGCUGAAACAGGCCGGCGACGUGGAGGAAAAUC
CUGGCCCUAUGGGCGCCGCCGCUAGCAUCCAGACAACAGUGAACACCCUG
AGCGAGCGGAUCAGCAGCAAGCUGGAGCAGGAGGCCAAUGCCUCUGCCCA
GACCAAAUGCGACAUCGAGAUCGGCAAUUUUUACAUCAGACAGAACCACG
GGUGCAACCUGACCGUGAAGAACAUGUGCUCCGCUGACGCGGAUGCCCAG
CUGGAUGCCGUCCUGUCUGCCGCCACUGAAACCUACAGCGGCCUGACACC
AGAACAAAAGGCCUACGUUCCUGCCAUGUUCACCGCCGCCCUGAACAUCC
AGACCUCUGUGAACACCGUGGUGCGGGACUUCGAGAAUUACGUGAAGCAG
ACAUGCAACAGCAGCGCUGUGGUGGACAACAAGCUGAAAAUUCAGAACGU
GAUCAUAGACGAGUGUUACGGAGCCCCUGGCAGCCCUACCAAUCUCGAGU
UCAUCAACACCGGCAGCAGCAAGGGCAAUUGCGCCAUCAAGGCCCUGAUG
CAGCUGACCACGAAGGCUACCACCCAGAUCGCCCCUAAGCAGGUGGCCGG
CACCGGCGUGCAGUUCUACAUGAUCGUGAUCGGCGUGAUCAUUCUGGCCG
CCCUCUUUAUGUACUACGCCAAGCGUAUGCUGUUCACCAGCACUAAUGAU
AAGAUCAAGCUGAUUCUGGCUAAUAAAGAGAACGUGCACUGGACCACAUA
UAUGGACACCUUCUUUAGAACCAGCCCCAUGGUGAUCGCCACCACCGACA
UGCAAAAUAGAAGAAAGAGGGGCUCUGGAGCCACCAACUUUAGCCUGCUG
AAGCAGGCUGGAGAUGUGGAAGAGAACCCUGGCCCUAUGAUGACACCUGA
GAACGAUGAGGAGCAAACCUCUGUGUUCAGCGCCACGGUGUACGGCGAUA
AGAUCCAGGGUAAGAAUAAGCGGAAGAGAGUGAUCGGCCUGUGUAUCCGG
AUCUCUAUGGUGAUCAGCUUGCUGAGCAUGAUCACCAUGUCUGCCUUCCU
GAUCGUGCGGCUGAACCAGUGCAUGUCCGCCAACGAAGCCGCCAUCACCG
ACGCCGCAGUGGCCGUGGCCGCAGCUAGCAGCACCCAUAGAAAAGUGGCA
AGCAGCACAACCCAGUACGAUCACAAAGAGUCCUGUAACGGCCUGUACUA
CCAAGGCUCUUGCUACAUCCUGCACAGCGACUAUCAGCUGUUCUCAGACG
CCAAGGCUAACUGCACUGCCGAAUCCAGCACCCUGCCUAACAAAAGCGAC
GUGCUGAUUACCUGGCUGAUCGACUACGUGGAGGACACCUGGGGCUCUGA
UGGCAACCCCAUCACUAAAACCACCAGCGAUUACCAGGACAGCGACGUCA
GCCAGGAGGUCAGAAAAUAUUUCUGCGUGAAGACCAUGAACCGGAGAAAG
AGAGGCAGCGGCGCCACCAACUUCUCACUGCUGAAGCAGGCCGGAGAUGU
GGAAGAAAACCCAGGCCCAAUGAAGACCAUCAGCGUGGUGACACUGCUGU
GCGUGCUCCCCGCUGUUGUGUACUCCACCUGUACAGUGCCUACAAUGAAC
AACGCCAAGCUGACAAGCACCGAGACCAGCUUCAACGAUAAGCAGAAGGU
GACCUUCACCUGUGACCAAGGAUACCACAGCCUGGACCCUAACGCUGUGU
GCGAGACAGACAAGUGGAAGUACGAGAACCCUUGUAAAAAGAUGUGUACA
GUGUCUGACUACGUGAGCGAGCUGUACGACAAGCCCCUGUACGAGGUCAA
UUCCACCAUGACCCUGAGCUGUAACGGCGAGACAAAGUACUUCAGAUGCG
AGGAAAAGAACGGCAACACAAGCUGGAACGACACCGUGACUUGCCCCAAC
GCCGAAUGCCAGCCGCUGCAGCUGGAGCACGGCAGCUGCCAGCCUGUCAA
AGAGAAGUAUAGCUUCGGUGAAUACAUUACAAUCAAUUGCGACGUGGGCU
ACGAGGUGAUCGGCGCCAGCUACAUCAGCUGUACCGCUAAUAGCUGGAAC
GUCAUCCCUUCCUGCCAGCAGAAGUGCGACAUGCCUUCUCUGUCUAACGG
CCUGAUCUCCGGAUCAACAUUUUCCAUCGGCGGCGUGAUCCACCUGAGCU
GCAAGUCCGGCUUCAUCCUGACCGGCUCCCCUUCUAGCACCUGCAUCGAU
GGCAAGUGGAACCCCAUCCUUCCAACCUGCGUGAGAUCCAACAAGGAGUU
CGACCCCGUGGACGACGGCCCCGACGACGAGACAGACCUCAGCAAGCUGA
GCAAGGAUGUGGUCCAGUACGAGCAGGAAAUCGAGAGCCUAGAGGCCACC
UACCACAUCAUCAUCGUGGCUCUGACAAUCAUGGGAGUGAUCUUCCUGAU
CUCUGUUAUCGUGCUGGUGUGCUCCUGUGACAAAAACAACGACCAGUACA
AGUUCCACAAGCUGCUGCCU
12. 5′UTR (containing Kozak)
(SEQ ID NO: 12)
GAGACUCUUCUGGUCCCCACAGACUCAGAGAGAACCCGCCACC
13. 3′UTR
(SEQ ID NO: 13)
GCUGGAGCCUCGGUGGCCAUGCUUCUUGCCCCUUGGGCCUCCCCAGCCCC
UCCUCCCCUUCGCACCCGUACCCCCGUGGUCUUUGAAUAAAGUCUGAGUG
GGGGGC
14. Poly A
(SEQ ID NO: 14)
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAA

### Specific Models for Carrying Out the Present Invention

The embodiments of the present invention will be described in detail with reference to the examples, but those skilled in the art will understand that the following examples are only used to illustrate the present invention and should not be regarded as limiting the scope of the present invention. If the specific conditions we not specified in the examples, they are carried out according to conventional conditions or conditions recommended by the manufacturer. The reagents or instruments used without indicating the manufacturer are all conventional products that can be purchased commercially.

### Preparation Example 1: Preparation of ALAB mRNA-LNP Vaccine

### 1. Sequence Design

The mRNA nucleic acid sequence (SEQ ID NO: 1) was 3100 nt in length ad consisted of the following components.
5′UTR sequence (1-43), CDS translation sequence (44-2863), stop codon (2864-2869), 3′UTR sequence (2870-2979) and PolyA sequence (2980-3100 nt). Among them, the 5′UTR sequence was the 5′UTR of Globin al plus the Kozak sequence and the 3′ sequence was the 3′UTR of Globin α1; the CDS sequence was composed of the coding nucleic acid sequences of four proteins, A27, L1, A33 and B5, and the Linker sequence; the stop codon was the TGATAA sequence; the PolyA sequence was 121 nt in length, which was obtained by ligating 60 As, 1 G and 60 As in series.

The protein sequence (SEQ ID NO: 2) in length of 940Aa encoded by CDS comprised A27. L1, A33, B5 and Linker; the amino acid length of A27 was 110Aa (1-110), the amino acid length of L1 was 250Aa (137-386), the amino acid length of A33 was 185Aa (413-597), and the amino acid length of B5 was 317Aa (624-940). The three Linkers were the same, and had an amino acid length of 26Aa (111-136, 387-412, 598-623).

Through sequence design, the coding region for tandem expression of four proteins A27, L1, A33 and B5 was obtained, named ALAB region, which was set forth in SEQ ID NO: 3; through human codon optimization, SEQ ID NO: 4 was obtained; T7 promoter sequence and 5′UTR sequence (including Kozak sequence) were added upstream of SEQ ID NO: 4, and the termination codon TGATAA sequence, 3′UTR sequence and PolyA sequence as well as BspQI restriction site were added downstream to obtain SEQ ID NO: 3.

### 2. Construction of Recombinant Plasmid

Gene synthesis of SEQ ID NO: 5 was caried out, and it was cloned into pUCYH plasmid (other cloning plasmids could also be used) by recombination; the resultant recombinant plasmid was named ABLB-pUCYH plasmid, and the sequence was verified to be correct by sequencing.

3. Preparation and Purification of ALAB mRNA

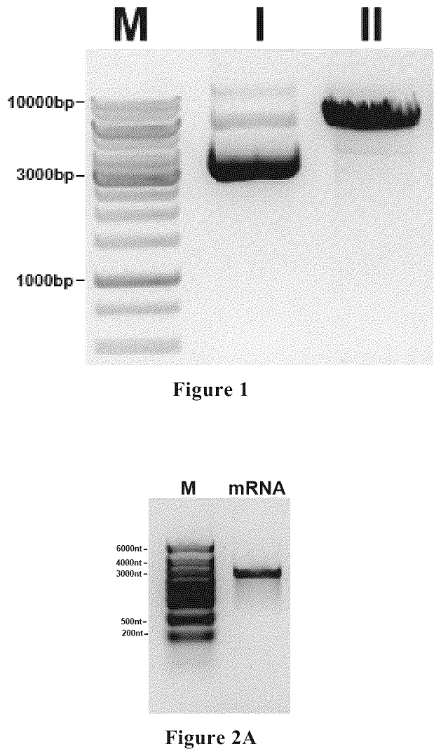

The recombinant plasmid ABLB-pUC57 plasmid was taken, enzymatically digested with restriction endonuclease BspQI, and the linearized plasmid was recovered and purified. The electrophoresis diagrams of the plasmid before (I) and after (II) enzyme digestion we shown in FIG. 1, wherein M denotes marker. I denotes the plasmid before linearization, and II denotes the plasmid after linearization.

The in vitro transcription IVT reaction (50 μl volume) was performed under the following conditions:
2 μg/10 μl linearized plasmid template, 2.5 μl of ATP (100 mM, Hongene), 2.5 μl of GP (100 mM, Hongene), 2.5 pd of CTP (100 mM, Hongene), 2.5 μl of UTP (100 mM, Hongene), 2 μl of GAG cap analog (100 mM, Trillink), 2 μl of T7 RNA polymerase (200 U/μl, Vazyme), 5 μl of 10× T7 reaction solution (Vazyme), 2.5 μl of RNase inhibitor (40 U/μl, Vazyme), 1.25 μl of pyrophosphatase (0.1 U/μl, Vazyme), and 17.25 μl of RNase-free H2O (Invitrogen™, 10977015) were mixed well under vortexing and shaking, and reacted at 37° C. for 3 hours.

After the reaction was completed, 170 μl of RNase-free H₂O, 5 μl of DNaseI enzyme (NEB, M0303L) and 25 μl of 10× DNaseI enzyme reaction solution (NEB, M0303L) were added, and reacted at 37° C. for 20 minutes.

mRNA was purified using OligoDT beads (Vazyme, N401).

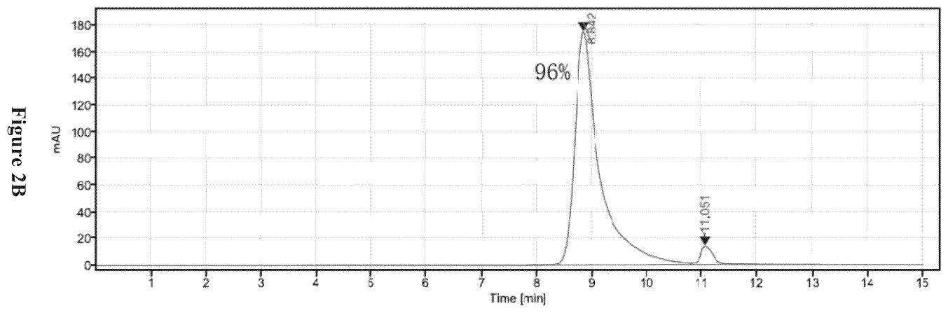

The gel electrophoresis results are shown in FIG. 2A. The results showed that the prepared mRNA had good purity and no obvious degradation.

The HPLC-SEC analysis results are shown in FIG. 2B. The results showed that the purity of the prepared mRNA could reach 96%.

4. Preparation of ALAB mRNA-LNP Vaccine Preparation

The purified mRNA was diluted to 167 μg/ml with 50 mM citrate buffer at pH4.0 to obtain n aqueous phase solution. Dlin-MC3-DMA (Avanti, 1224606-06-7), DSPC (Avanti, S01005), cholesterol (Sigma-Aldrich, C8667) and 14:0PEG2000-PE (Avanti Polar Lipids, 880150P) in certain amounts were dissolved in anhydrous ethanol to obtain an organic phase solution, in which the final concentration of Dlin-MC3-DMA was 6.25 mM, the final concentration of DSPC was 1.25 mM, the final concentration of cholesterol was 4.81 mM, and the final concentration of 14:0PEG2000-PE was 0.19 mM.

The aqueous phase solution and the organic phase solution were pipetted into the injection device of microfluidic apparatus (MicroNano, INano™ L), and the mRNA embedding process was performed at a flow rate ratio of 3:1 (aqueous phase: 3, organic phase: 1) and a total flow rate of 12 ml/min. and the effluent mRNA-LNP complex liquid was collected. The effluent was quickly diluted to 39 times its volume with cooled (4° C.) PBS at pH7.4, mixed well, and centrifuged for 10 minutes at 4° C., 3000 rmp using a 100 kD ultrafiltration tube (Millipore, UFC910024) to concentrate the mRNA complex to 0.4 mg/ml; it was then filtered using a 0.22 um filter membrane, the filtrate was collected to obtain an ALAB mRNA-LNP vaccine preparation.

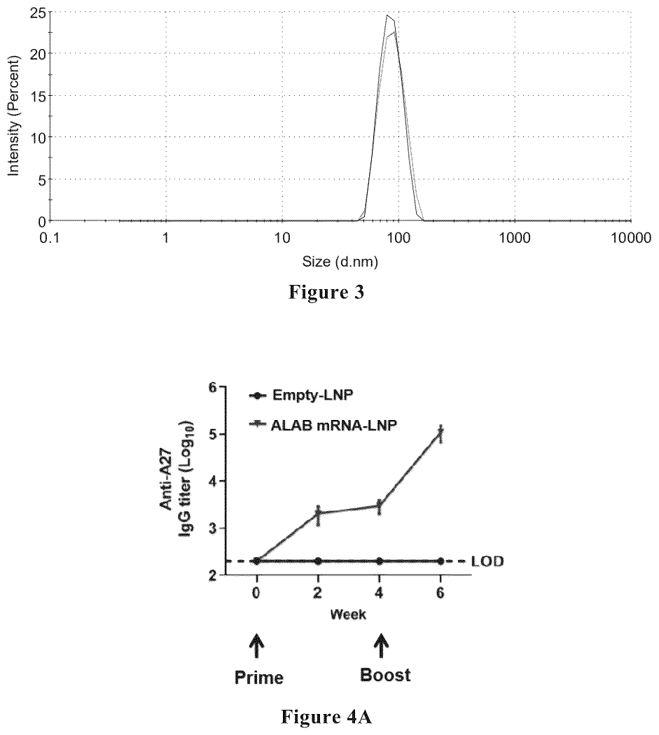

The ALAB mRNA-LNP vaccine preparation was analyzed using a Malvern particle size analyzer, and the average particle size of the preparation was 84.45 nm (see FIG. 3), and PDI <0.1. The mRNA encapsulation efficiency of the preparation was measured by the RiboGreen (Invitrogen™, R11490) nucleic acid dye method to be 98.1%.

### Experimental Example 1: Evaluation of Immunogenicity of ALAB mRNA-LNP Vaccine

### 1. Design of Immunization Scheme

Balb/c nice aged 6-8 weeks were grouped, 5 mice in each group, into an empty group (Empty-LNP) and a control group (ALAB mRNA-LNP); the entire experimental cycle was carried out in the SPF-level mouse room of Bikai Laboratory Animal Co., Ltd.

The day of the first immunization of mice was defined as Day 0, the day before immunization was defined as Day −1, the day after immunization was defined a Day 1, and so on. The prepared vaccine was injected intramuscularly on Day 0 and Day 28 to immunize mice, with each mouse receiving a dose of 20 μl/60 μl per immunization.

Blood samples were collected from mice on Day −1 and every 2 weeks after the first immunization, and the sera were separated and stored in a −80° C. refrigerator for antigen-specific antibody detection and heterologous antigen-antibody cross-reaction. Mouse spleens were taken 35 days after the second immunization for cellular immunity evaluation.

### 2. Detection of VACV Antigen-Specific Binding Antibodies

The ELISA method was used to detect the titer of antigen-specific antibody IgG in mouse serum during the entire immunization cycle. The specific steps were as follows:

The four antigens A27 (CUSABIO, CSB-EP31812OVAI), L1 (CUSABIO, CSB-EP324949VAA1), A33 (CUSABIO, CSB-EP300755VAA1), and B5 (CUSABIO, CSB-EP321891VAA1) of VACV were separately diluted to 1 μg/ml with 1× ELISA coating solution (Solarbio, C1055), and the above dilution solutions were added to a 96-well ELISA plate (Corning, 9018) at 100 μl/well, and allowed to stand at 4° C. overnight for coating. The liquid in the plate was discarded, the plate was washed with PBST three times, then 5% skim milk powder prepared with PBST was added at 200 μl/well, and allowed to stand at 37° C. for 1 hour for blocking. Then the plate was washed three times with PBST. During the blocking period, the mouse sera were diluted with PBST containing 2% skim milk powder. The sera collected before immunization, 2 weeks after the first immunization, and 4 weeks after the first immunization were separately diluted from 1:200, and then 3-fold gradient dilution was performed, for a total of 8 gradients. The sera collected 2 weeks after the second immunization was diluted from 1:10000, and then 3-fold gradient dilution was performed as well, for a total of 8 gradients. The above diluted mouse sera were added to the blocked and well-washed plate at 100 μl/well, incubated at 37° C. for 1 hour, and then the plate was washed 5 times with PBST. Goat anti-mouse IgG-HRP (SouthernBiotech, 1031-05) diluted at 1:5000 with PBST containing 2% skim milk powder was added at 100 μl/well, incubated at 37° C. for 1 hour, and then the plate was washed 5 times with PBST. TMB colorimetric solution (Invitrogen™, 002023) was added at 100 μl/well, and allowed to stand at room temperature for 3 minutes. ELISA stop solution (New Cyme, E40500) was added at 50 μl/well. Detection was performed at OD450 wavelength using a microplate reader (Thermo Fisher, Varioskan LUX), and the serum antibody titer was finally determined based on 2.1 times the blank serum reading as the cut-off value.

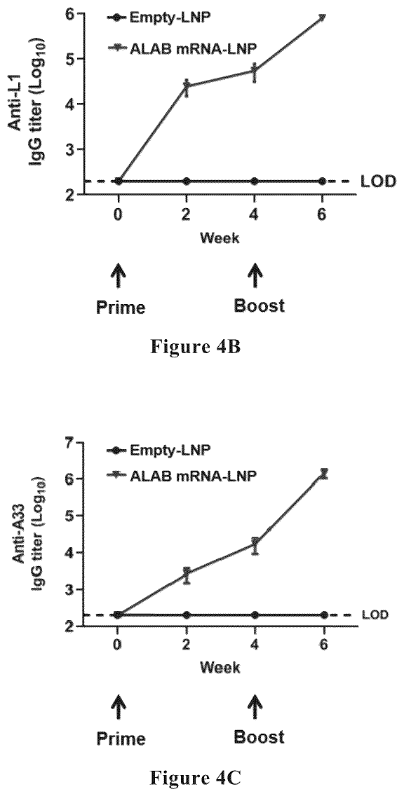

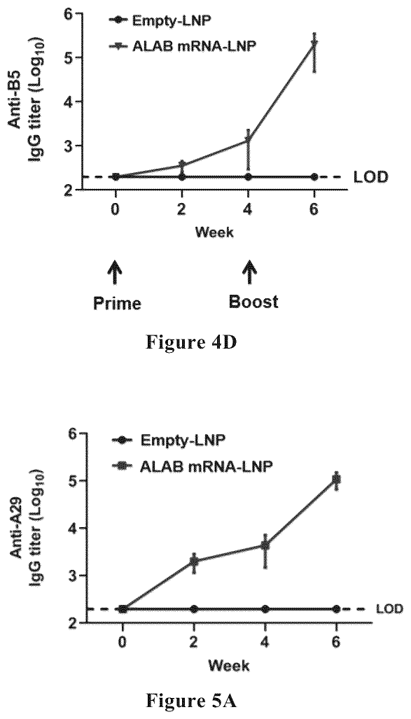

The results are shown in FIGS. 4A to 4D.

The results showed that the ALAB mRNA-LNP vaccine could induce high levels of specific binding antibodies against each of the antigens (A27, A33, L1 and B5) in mice. And the antibody levels against the four antigens were significantly improved after the second immunization.

### 3. Detection of MPXV Antigen Cross-Binding Antibodies

The proteins in the monkeypox virus MPXV that are homologous to A27, L1, A33 and B5 of vaccinia virus are MPXV A29, M1, A35 and B6, respectively. The conservation of the antigens is 94% to 99%, and studies have shown that the corresponding four antigens of the two viruses have the potential for cross-immunity. In order to study the cross-binding reaction of ALAB mRNA-LNP vaccine mouse immune serum to the four antigens A29 (Oukai Biotech, C1618). M1 (nearshore protein, DRA210), A35 (nearshore protein, DRA209) and B6 (nearshore protein, DRA211) of MPXV, the ELISA method was used to detect the antigen-specific antibody IgG titers of A29, M1, A35 and B6 in mouse serum. The specific steps were as follows: The four antigens A29, M1, A35 mid B6 of MPXV were diluted to 1 μg/ml with 1× ELISA coating solution (Solarbio, C1055), respectively, and the above dilution solutions were added to a 96-well ELISA plate (Corning, 9018) at 100 μl/well, and allowed to stand at 4° C. overnight for coating. The liquid in the well plate was discarded, the plate was washed 3 times with PBST, then 5% skim milk powder prepared with PBST was added at 200 μl/well, and allowed to stand at 37° C. for 1 hour for blocking. Then the plate was washed 3 times with PBST. During the blocking period, the mouse sera were diluted with PBST containing 2% skim milk powder, and in which the sera collected before immunization, 2 weeks after the first immunization, and 4 weeks after the first immunization were diluted from 1:200, and then 3-fold gradient dilution was performed, for a total of 8 gradients. The sera collected 2 weeks after the second immunization were diluted from 1:10000, and 3-fold gradient dilution was performed as well, for a total of 8 gradients. The above diluted mouse sera were separately added the blocked and well-washed plate at 100 μl/well, and incubated at 37° C. for 1 hour, and then the plate s washed 5 times with PBST. Goat anti-mouse IgG-HRP (SouthernBiotech, 1031-05) diluted at 1:000 in PBST containing 2% skim milk powder was added at 100 μl/well, and incubated at 37° C. for 1 hour, then the plate was washed 5 times with PBST. TMB colorimetric solution (Invitrogen™, 002023) was added at 100 μl/well, ad incubated at room temperature for 3 minutes. ELISA stop solution (New Cyme, E40500) was added at 50 μl/well. Detection was performed at OD450 wavelength using a microplate reader (Thermo Fisher, Varioskan LUX), and the serum antibody titer was finally determined based on 2.1 times the blank serum reading as the cut-off value.

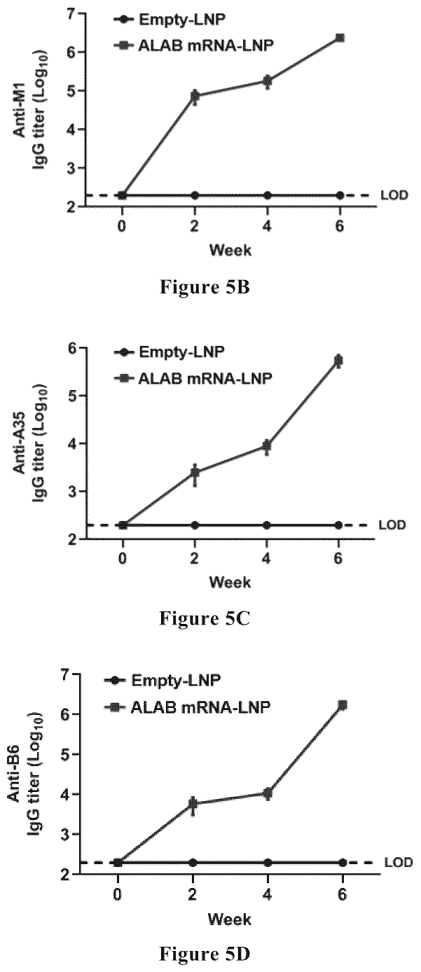

The results me shown in FIGS. 5A to 5D.

The results showed that the ALAB mRNA-LNP vaccine could induce high levels of cross-binding antibodies against each of the MPXV antigens (A29, A35, M1 and B6) in mice. And the antibody levels were significantly improved after the second immunization.

### 4. Detection of Neutralizing Antibody Titer

In order to detect the neutralizing antibody content in the vaccine-induced serum antibodies, the mouse sera were tested 14 days after the second immunization. The specific operation was as follows:

After the sera were inactivated at 56° C. for 30 minutes, 2-fold gradient dilution was performed from 1:40 with serum-free MEM medium (Gibco), for a total of 10 gradients. Then 50 μl of serum was mixed with an equal volume of VACV (100 TCID50) and incubated at 37° C. for 2 hours. The mixed solution after incubation was added to a 96-well cell plate coated on the day before with BSC-1 (1×10⁴/well), and incubated at 37° C. for 3 days, then the CPE and cell growth status were observed to determine the number of virus-infected cells and calculate the infection rate at each serum dilution. Finally. The serum neutralizing antibody titer was finally obtained according to the formula.

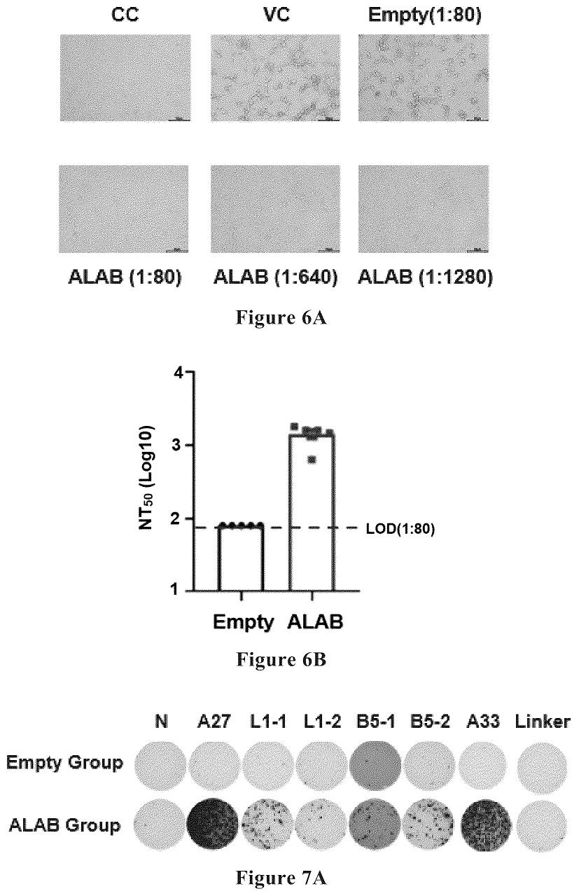

The results are shown in FIGS. 6A to 6B.

The results showed that the cells in the virus-free negative cell wells (CC) were normal and had no obvious lesions. The cells in the serum-free virus infection well (VC) and the empty serum infection group (Empty) showed obvious cytopathic effects ad no obvious neutralizing activity. ALAB mRNA-LNP vaccine induced obvious neutralizing antibodies after immunization, and the final neutralizing antibody titer was greater than 1000.

### 5. Detection of Cellular Immune Response

35% Ethanol was added at 100p/well to an ELISPOT plate (Millipore, MSIPS4W10). The liquid was discarded after 1 minute, and the plate was washed 3 times with sterile deionized water at 200 μl/well. IFN-γ capture antibody (MABTECH, 3321-2H) was diluted with PBS to 15 μg/ml, added to the above plate at 100 μl/well, and incubated at 4° C. overnight. On the next day, the plate was washed with PBS, then added with RPMI 1640 (Gibco, 61870127)+10% FBS (Gibco, 10091148) and incubated at room temperature for 30 minutes. The spleens of the mice were taken and single cell suspensions were prepared. The lysis of red cells was performed, and counting was carried out after washing with PBS. The medium in the ELISPOT plate was discarded, and an appropriate amount of the cells were added. At the same time, peptide libraries (one peptide library for A27, two peptide libraries (namely L1-1 and L1-2) for L1, one peptide library for A33, and two peptide libraries (namely B35-1 and B5-2) for B5) synthesized with different antigens were added, so that the final concentration of each peptide was 2.5 μg/ml. After the well plate was allowed to stand in a 37° C. cell culture incubator for 36 hours, the cells were discarded, and the plate was washed with PBS. Thien the detection antibody (MABTECH, 3321-2H) as diluted (1:1000) was added, and incubated at room temperature for 2 hours. After the above operation was completed, the plate was washed again with PBS, added with diluted (1:1000) streptavidin-HRP (MABTECH, 3321-2H), and incubated at room temperature for 1 hour. After the plate was washed with PBS, TMB colorimetric solution (MABTECH, 3631-10) was added, and then the plate was washed with deionized water after spots were obvious. The plate was dried at room temperature, then pictured using a CTL enzyme-linked immunospot analyzer (CTL. S6 Universal), and the spots in wells were counted.

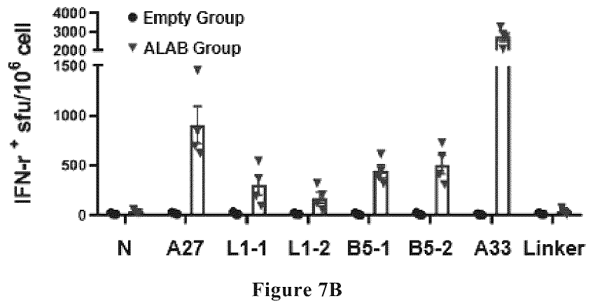

The results are shown in FIGS. 7A to 7B.

The results showed that there was no obvious spot formation in the spleen cells of the empty control group after stimulation with different antigenic peptides, while different numbers of spots would be produced in the ALAB mRNA-LNP vaccine immunization group after stimulation with different antigenic peptides, indicating that the vaccine induced four antigen-specific cellular immune responses in mice.

Although the specific embodiments of the present invention have been described in detail, those skilled in the art will understand that various modifications and substitutions can be made to those details based on all the teachings that have been disclosed, and these changes are within the scope of protection of the present invention. The full scope of the present invention is given by the appended claims and any equivalents thereof.

1. An isolated mRNA molecule, which comprises a coding region, wherein the coding region encodes a target protein, and the target protein comprises the following four proteins:
vaccinia virus surface antigens A27, L1, A33 and B5;
wherein, two adjacent proteins are independently connected directly or connected via identical or different Linkers.

2. The isolated mRNA molecule according to claim 1, wherein:
A27 has an amino acid sequence as set forth in SEQ ID NO: 6,
L1 has an amino acid sequence as set forth in SEQ ID NO: 7,
A33 has an amino acid sequence as set forth in SEQ ID NO: 8, and/or
B5 has an amino acid sequence as set forth in SEQ ID NO: 9.

3. The isolated mRNA molecule according to claim 1, wherein in the target protein, A27, L1, A33 and B5 are arranged in sequence;
preferably, in the target protein, A27, Linker, L1, Linker, A33, Linker and B5 are arranged in sequence.

4. The isolated mRNA molecule according to claim 1, wherein the Linker has an amino acid sequence as set forth in SEQ ID NO: 10.

5. The isolated mRNA molecule according to claim 1, wherein the target protein has an amino acid sequence as set forth in SEQ ID NO: 2.

6. The isolated mRNA molecule according to claim 1, wherein the coding region has a sequence as set forth in SEQ ID NO: 11.

7. The isolated mRNA molecule according to claim 1, which further comprises one or more selected from the following:
5UTR, stop codon, 3′UTR and PolyA;
preferably, the isolated mRNA molecule comprises in sequence:
5′UTR, coding region, stop codon, 3′UTR and PolyA.

8. The isolated mRNA molecule according to claim 7, wherein:
5′UTR has a sequence as set forth in SEQ ID NO: 12,
the stop codon has a sequence of UGAUAA,
3′UTR has a sequence as set forth in SEQ ID NO: 13, and/or
PolyA has a sequence as set forth in SEQ ID NO: 14.

9. The isolated mRNA molecule according to claim 1, which has a sequence as set forth in SEQ ID NO: 1.

10. An isolated DNA molecule, which is capable of transcribing the isolated mRNA molecule according to claim 1.

11. The isolated DNA molecule according to claim 10, which comprises a DNA fragment having a sequence as set forth in SEQ ID NO: 3 or SEQ ID NO: 4.

12. The isolated DNA molecule according to claim 10, which further comprises an upstream promoter, preferably a T7 promoter.

13. The isolated DNA molecule according to claim 10, which has a sequence as set forth in SEQ ID NO: 5.

14. A recombinant plasmid, which comprises the isolated DNA molecule according to claim 10.

15. A recombinant host cell, which comprises the isolated DNA molecule according to claim 10 or a recombinant plasmid comprising the isolated DNA molecule according to claim 10.

16. An mRNA vaccine preparation, which comprises the isolated mRNA molecule according to claim 1, and an mRNA vaccine vector;
preferably, the mRNA vaccine vector is a lipid nanoparticle or a polymer nanoparticle;
preferably, the lipid nanoparticle;
preferably, the mRNA vaccine preparation has an average particle size of 50-100 nm, 70-90 nm, 80-90 nm or 85 nm.

17. The mRNA vaccine preparation according to claim 16, which is a vaccine preparation for preventing or treating a variola or monkeypox virus infection.

18.-19. (canceled)

20. A method for preventing or treating variola or monkeypox virus infection, comprising a step of administering to a subject in need thereof an effective amount of the isolated mRNA molecule according to claim 1.


SEQUENCE LISTING (14 sequences)

SEQ ID NO: 1 - RNA, 3100 nt, synthetic construct
  source 1..3100: mol_type = other RNA
gagactcttctggtccccacagactcagagagaacccgccaccatggacggaactctgttccccggcgacgacgatctggccatccccgccaccgagtttttcagcacaaaggccgctaagaagcctgaagccaagagagaggccatcgtgaaggccgatgaagatgataacgaggaaaccctgaagcagagactgacaaatctcgaaaagaagatcaccaacgtgaccacaaagttcgagcaaattgaaaagtgctgcaagcggaacgatgaagttctattccggctggaaaaccacgccgagaccctgagagccgccatgatcagcctggccaagaaaatcgacgtgcaaacaggcagacggccttacgaaagaagaaagaggggcagcggcgccacaaacttctcactgctgaaacaggccggcgacgtggaggaaaatcctggccctatgggcgccgccgctagcatccagacaacagtgaacaccctgagcgagcggatcagcagcaagctggagcaggaggccaatgcctctgcccagaccaaatgcgacatcgagatcggcaatttttacatcagacagaaccacgggtgcaacctgaccgtgaagaacatgtgctccgctgacgcggatgcccagctggatgccgtcctgtctgccgccactgaaacctacagcggcctgacaccagaacaaaaggcctacgttcctgccatgttcaccgccgccctgaacatccagacctctgtgaacaccgtggtgcgggacttcgagaattacgtgaagcagacatgcaacagcagcgctgtggtggacaacaagctgaaaattcagaacgtgatcatagacgagtgttacggagcccctggcagccctaccaatctcgagttcatcaacaccggcagcagcaagggcaattgcgccatcaaggccctgatgcagctgaccacgaaggctaccacccagatcgcccctaagcaggtggccggcaccggcgtgcagttctacatgatcgtgatcggcgtgatcattctggccgccctctttatgtactacgccaagcgtatgctgttcaccagcactaatgataagatcaagctgattctggctaataaagagaacgtgcactggaccacatatatggacaccttctttagaaccagccccatggtgatcgccaccaccgacatgcaaaatagaagaaagaggggctctggagccaccaactttagcctgctgaagcaggctggagatgtggaagagaaccctggccctatgatgacacctgagaacgatgaggagcaaacctctgtgttcagcgccacggtgtacggcgataagatccagggtaagaataagcggaagagagtgatcggcctgtgtatccggatctctatggtgatcagcttgctgagcatgatcaccatgtctgccttcctgatcgtgcggctgaaccagtgcatgtccgccaacgaagccgccatcaccgacgccgcagtggccgtggccgcagctagcagcacccatagaaaagtggcaagcagcacaacccagtacgatcacaaagagtcctgtaacggcctgtactaccaaggctcttgctacatcctgcacagcgactatcagctgttctcagacgccaaggctaactgcactgccgaatccagcaccctgcctaacaaaagcgacgtgctgattacctggctgatcgactacgtggaggacacctggggctctgatggcaaccccatcactaaaaccaccagcgattaccaggacagcgacgtcagccaggaggtcagaaaatatttctgcgtgaagaccatgaaccggagaaagagaggcagcggcgccaccaacttctcactgctgaagcaggccggagatgtggaagaaaacccaggcccaatgaagaccatcagcgtggtgacactgctgtgcgtgctccccgctgttgtgtactccacctgtacagtgcctacaatgaacaacgccaagctgacaagcaccgagaccagcttcaacgataagcagaaggtgaccttcacctgtgaccaaggataccacagcctggaccctaacgctgtgtgcgagacagacaagtggaagtacgagaacccttgtaaaaagatgtgtacagtgtctgactacgtgagcgagctgtacgacaagcccctgtacgaggtcaattccaccatgaccctgagctgtaacggcgagacaaagtacttcagatgcgaggaaaagaacggcaacacaagctggaacgacaccgtgacttgccccaacgccgaatgccagccgctgcagctggagcacggcagctgccagcctgtcaaagagaagtatagcttcggtgaatacattacaatcaattgcgacgtgggctacgaggtgatcggcgccagctacatcagctgtaccgctaatagctggaacgtcatcccttcctgccagcagaagtgcgacatgccttctctgtctaacggcctgatctccggatcaacattttccatcggcggcgtgatccacctgagctgcaagtccggcttcatcctgaccggctccccttctagcacctgcatcgatggcaagtggaaccccatccttccaacctgcgtgagatccaacaaggagttcgaccccgtggacgacggccccgacgacgagacagacctcagcaagctgagcaaggatgtggtccagtacgagcaggaaatcgagagcctagaggccacctaccacatcatcatcgtggctctgacaatcatgggagtgatcttcctgatctctgttatcgtgctggtgtgctcctgtgacaaaaacaacgaccagtacaagttccacaagctgctgccttgataagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaa

SEQ ID NO: 2 - protein, 940 aa, synthetic construct
MDGTLFPGDDDLAIPATEFFSTKAAKKPEAKREAIVKADEDDNEETLKQRLTNLEKKITNVTTKFEQIEKCCKRNDEVLFRLENHAETLRAAMISLAKKIDVQTGRRPYERRKRGSGATNFSLLKQAGDVEENPGPMGAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPKQVAGTGVQFYMIVIGVIILAALFMYYAKRMLFTSTNDKIKLILANKENVHWTTYMDTFFRTSPMVIATTDMQNRRKRGSGATNFSLLKQAGDVEENPGPMMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDVSQEVRKYFCVKTMNRRKRGSGATNFSLLKQAGDVEENPGPMKTISVVTLLCVLPAVVYSTCTVPTMNNAKLTSTETSFNDKQKVTFTCDQGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDMPSLSNGLISGSTFSIGGVIHLSCKSGFILTGSPSSTCIDGKWNPILPTCVRSNKEFDPVDDGPDDETDLSKLSKDVVQYEQEIESLEATYHIIIVALTIMGVIFLISVIVLVCSCDKNNDQYKFHKLLP

SEQ ID NO: 3 - DNA, 2820 bp, synthetic construct
  source 1..2820: mol_type = other DNA
atggacggaactcttttccccggagatgacgatcttgcaattccagcaactgaatttttttctacaaaggctgctaaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgagcgcagaaagagaggctccggcgccacaaacttcagcctgctgaagcaggctggcgacgtcgaggaaaaccccggccctatgggtgccgcagcaagcatacagacgacggtgaatacactcagcgaacgtatctcgtctaaattagaacaagaagcgaacgctagtgctcaaacaaaatgtgatatagaaatcggaaatttttatatccgacaaaaccatggatgtaacctcactgttaaaaatatgtgctctgcggacgcggatgctcagttggatgctgtgttatcagccgctacagaaacatatagtggattaacaccggaacaaaaagcatacgtaccagctatgtttactgctgcgttaaacattcagacaagtgtaaacactgttgttagagattttgaaaattatgtgaaacaaacttgtaattctagcgcggtcgtcgataacaaattaaagatacaaaacgtaatcatagatgaatgttacggagccccaggatctccaacaaatttggaatttattaatacaggatctagcaaaggaaattgtgccattaaggcgttgatgcaattgactactaaggccactactcaaatagcacctaaacaagttgctggtacaggagttcagttttatatgattgttatcggtgttataatattggcagcgttgtttatgtactatgccaagcgtatgctgttcacatccaccaatgataaaatcaaacttattttagccaataaggaaaacgtccattggactacttacatggacacattctttagaacttctccgatggttattgctaccacggatatgcaaaaccgcagaaagagaggctccggcgccacaaacttcagcctgctgaagcaggctggcgacgtcgaggaaaaccccggccctatgatgacaccagaaaacgacgaagagcagacatctgtgttctccgctactgtttacggagacaaaattcagggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacacaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatgtatcacaagaagttagaaagtatttttgtgttaaaacaatgaaccgcagaaagagaggctccggcgccacaaacttcagcctgctgaagcaggctggcgacgtcgaggaaaaccccggccctatgaaaacgatttccgttgttacgttgttatgcgtactacctgctgttgtttattcaacatgtactgtacccactatgaataacgctaaattaacgtctaccgaaacatcgtttaatgataaacagaaagttacatttacatgtgatcagggatatcattctttggatccaaatgctgtctgcgaaacagataaatggaaatacgaaaatccatgcaagaaaatgtgcacagtttctgattatgtctctgaattatatgataagccattatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatataactatcaactgtgatgttggatatgaggttattggtgcttcgtacataagttgtacagctaattcttggaatgttattccatcatgtcaacaaaaatgtgatatgccgtctctatctaacggattaatttccggatctacattttctatcggtggcgttatacatcttagttgtaaaagtggttttatactaacgggatctccatcatccacatgtatcgacggtaaatggaatcccatactcccaacatgtgtacgatctaacaaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccg

SEQ ID NO: 4 - DNA, 2820 bp, synthetic construct
  source 1..2820: mol_type = other DNA
atggacggaactctgttccccggcgacgacgatctggccatccccgccaccgagtttttcagcacaaaggccgctaagaagcctgaagccaagagagaggccatcgtgaaggccgatgaagatgataacgaggaaaccctgaagcagagactgacaaatctcgaaaagaagatcaccaacgtgaccacaaagttcgagcaaattgaaaagtgctgcaagcggaacgatgaagttctattccggctggaaaaccacgccgagaccctgagagccgccatgatcagcctggccaagaaaatcgacgtgcaaacaggcagacggccttacgaaagaagaaagaggggcagcggcgccacaaacttctcactgctgaaacaggccggcgacgtggaggaaaatcctggccctatgggcgccgccgctagcatccagacaacagtgaacaccctgagcgagcggatcagcagcaagctggagcaggaggccaatgcctctgcccagaccaaatgcgacatcgagatcggcaatttttacatcagacagaaccacgggtgcaacctgaccgtgaagaacatgtgctccgctgacgcggatgcccagctggatgccgtcctgtctgccgccactgaaacctacagcggcctgacaccagaacaaaaggcctacgttcctgccatgttcaccgccgccctgaacatccagacctctgtgaacaccgtggtgcgggacttcgagaattacgtgaagcagacatgcaacagcagcgctgtggtggacaacaagctgaaaattcagaacgtgatcatagacgagtgttacggagcccctggcagccctaccaatctcgagttcatcaacaccggcagcagcaagggcaattgcgccatcaaggccctgatgcagctgaccacgaaggctaccacccagatcgcccctaagcaggtggccggcaccggcgtgcagttctacatgatcgtgatcggcgtgatcattctggccgccctctttatgtactacgccaagcgtatgctgttcaccagcactaatgataagatcaagctgattctggctaataaagagaacgtgcactggaccacatatatggacaccttctttagaaccagccccatggtgatcgccaccaccgacatgcaaaatagaagaaagaggggctctggagccaccaactttagcctgctgaagcaggctggagatgtggaagagaaccctggccctatgatgacacctgagaacgatgaggagcaaacctctgtgttcagcgccacggtgtacggcgataagatccagggtaagaataagcggaagagagtgatcggcctgtgtatccggatctctatggtgatcagcttgctgagcatgatcaccatgtctgccttcctgatcgtgcggctgaaccagtgcatgtccgccaacgaagccgccatcaccgacgccgcagtggccgtggccgcagctagcagcacccatagaaaagtggcaagcagcacaacccagtacgatcacaaagagtcctgtaacggcctgtactaccaaggctcttgctacatcctgcacagcgactatcagctgttctcagacgccaaggctaactgcactgccgaatccagcaccctgcctaacaaaagcgacgtgctgattacctggctgatcgactacgtggaggacacctggggctctgatggcaaccccatcactaaaaccaccagcgattaccaggacagcgacgtcagccaggaggtcagaaaatatttctgcgtgaagaccatgaaccggagaaagagaggcagcggcgccaccaacttctcactgctgaagcaggccggagatgtggaagaaaacccaggcccaatgaagaccatcagcgtggtgacactgctgtgcgtgctccccgctgttgtgtactccacctgtacagtgcctacaatgaacaacgccaagctgacaagcaccgagaccagcttcaacgataagcagaaggtgaccttcacctgtgaccaaggataccacagcctggaccctaacgctgtgtgcgagacagacaagtggaagtacgagaacccttgtaaaaagatgtgtacagtgtctgactacgtgagcgagctgtacgacaagcccctgtacgaggtcaattccaccatgaccctgagctgtaacggcgagacaaagtacttcagatgcgaggaaaagaacggcaacacaagctggaacgacaccgtgacttgccccaacgccgaatgccagccgctgcagctggagcacggcagctgccagcctgtcaaagagaagtatagcttcggtgaatacattacaatcaattgcgacgtgggctacgaggtgatcggcgccagctacatcagctgtaccgctaatagctggaacgtcatcccttcctgccagcagaagtgcgacatgccttctctgtctaacggcctgatctccggatcaacattttccatcggcggcgtgatccacctgagctgcaagtccggcttcatcctgaccggctccccttctagcacctgcatcgatggcaagtggaaccccatccttccaacctgcgtgagatccaacaaggagttcgaccccgtggacgacggccccgacgacgagacagacctcagcaagctgagcaaggatgtggtccagtacgagcaggaaatcgagagcctagaggccacctaccacatcatcatcgtggctctgacaatcatgggagtgatcttcctgatctctgttatcgtgctggtgtgctcctgtgacaaaaacaacgaccagtacaagttccacaagctgctgcct

SEQ ID NO: 5 - DNA, 3124 bp, synthetic construct
  source 1..3124: mol_type = other DNA
taatacgactcactataagactcttctggtccccacagactcagagagaacccgccaccatggacggaactctgttccccggcgacgacgatctggccatccccgccaccgagtttttcagcacaaaggccgctaagaagcctgaagccaagagagaggccatcgtgaaggccgatgaagatgataacgaggaaaccctgaagcagagactgacaaatctcgaaaagaagatcaccaacgtgaccacaaagttcgagcaaattgaaaagtgctgcaagcggaacgatgaagttctattccggctggaaaaccacgccgagaccctgagagccgccatgatcagcctggccaagaaaatcgacgtgcaaacaggcagacggccttacgaaagaagaaagaggggcagcggcgccacaaacttctcactgctgaaacaggccggcgacgtggaggaaaatcctggccctatgggcgccgccgctagcatccagacaacagtgaacaccctgagcgagcggatcagcagcaagctggagcaggaggccaatgcctctgcccagaccaaatgcgacatcgagatcggcaatttttacatcagacagaaccacgggtgcaacctgaccgtgaagaacatgtgctccgctgacgcggatgcccagctggatgccgtcctgtctgccgccactgaaacctacagcggcctgacaccagaacaaaaggcctacgttcctgccatgttcaccgccgccctgaacatccagacctctgtgaacaccgtggtgcgggacttcgagaattacgtgaagcagacatgcaacagcagcgctgtggtggacaacaagctgaaaattcagaacgtgatcatagacgagtgttacggagcccctggcagccctaccaatctcgagttcatcaacaccggcagcagcaagggcaattgcgccatcaaggccctgatgcagctgaccacgaaggctaccacccagatcgcccctaagcaggtggccggcaccggcgtgcagttctacatgatcgtgatcggcgtgatcattctggccgccctctttatgtactacgccaagcgtatgctgttcaccagcactaatgataagatcaagctgattctggctaataaagagaacgtgcactggaccacatatatggacaccttctttagaaccagccccatggtgatcgccaccaccgacatgcaaaatagaagaaagaggggctctggagccaccaactttagcctgctgaagcaggctggagatgtggaagagaaccctggccctatgatgacacctgagaacgatgaggagcaaacctctgtgttcagcgccacggtgtacggcgataagatccagggtaagaataagcggaagagagtgatcggcctgtgtatccggatctctatggtgatcagcttgctgagcatgatcaccatgtctgccttcctgatcgtgcggctgaaccagtgcatgtccgccaacgaagccgccatcaccgacgccgcagtggccgtggccgcagctagcagcacccatagaaaagtggcaagcagcacaacccagtacgatcacaaagagtcctgtaacggcctgtactaccaaggctcttgctacatcctgcacagcgactatcagctgttctcagacgccaaggctaactgcactgccgaatccagcaccctgcctaacaaaagcgacgtgctgattacctggctgatcgactacgtggaggacacctggggctctgatggcaaccccatcactaaaaccaccagcgattaccaggacagcgacgtcagccaggaggtcagaaaatatttctgcgtgaagaccatgaaccggagaaagagaggcagcggcgccaccaacttctcactgctgaagcaggccggagatgtggaagaaaacccaggcccaatgaagaccatcagcgtggtgacactgctgtgcgtgctccccgctgttgtgtactccacctgtacagtgcctacaatgaacaacgccaagctgacaagcaccgagaccagcttcaacgataagcagaaggtgaccttcacctgtgaccaaggataccacagcctggaccctaacgctgtgtgcgagacagacaagtggaagtacgagaacccttgtaaaaagatgtgtacagtgtctgactacgtgagcgagctgtacgacaagcccctgtacgaggtcaattccaccatgaccctgagctgtaacggcgagacaaagtacttcagatgcgaggaaaagaacggcaacacaagctggaacgacaccgtgacttgccccaacgccgaatgccagccgctgcagctggagcacggcagctgccagcctgtcaaagagaagtatagcttcggtgaatacattacaatcaattgcgacgtgggctacgaggtgatcggcgccagctacatcagctgtaccgctaatagctggaacgtcatcccttcctgccagcagaagtgcgacatgccttctctgtctaacggcctgatctccggatcaacattttccatcggcggcgtgatccacctgagctgcaagtccggcttcatcctgaccggctccccttctagcacctgcatcgatggcaagtggaaccccatccttccaacctgcgtgagatccaacaaggagttcgaccccgtggacgacggccccgacgacgagacagacctcagcaagctgagcaaggatgtggtccagtacgagcaggaaatcgagagcctagaggccacctaccacatcatcatcgtggctctgacaatcatgggagtgatcttcctgatctctgttatcgtgctggtgtgctcctgtgacaaaaacaacgaccagtacaagttccacaagctgctgccttgataagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaaagaagagc

SEQ ID NO: 6 - protein, 110 aa, synthetic construct
MDGTLFPGDDDLAIPATEFFSTKAAKKPEAKREAIVKADEDDNEETLKQRLTNLEKKITNVTTKFEQIEKCCKRNDEVLFRLENHAETLRAAMISLAKKIDVQTGRRPYE

SEQ ID NO: 7 - protein, 250 aa, synthetic construct
MGAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPKQVAGTGVQFYMIVIGVIILAALFMYYAKRMLFTSTNDKIKLILANKENVHWTTYMDTFFRTSPMVIATTDMQN

SEQ ID NO: 8 - protein, 185 aa, synthetic construct
MMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDVSQEVRKYFCVKTMN

SEQ ID NO: 9 - protein, 317 aa, synthetic construct
MKTISVVTLLCVLPAVVYSTCTVPTMNNAKLTSTETSFNDKQKVTFTCDQGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDMPSLSNGLISGSTFSIGGVIHLSCKSGFILTGSPSSTCIDGKWNPILPTCVRSNKEFDPVDDGPDDETDLSKLSKDVVQYEQEIESLEATYHIIIVALTIMGVIFLISVIVLVCSCDKNNDQYKFHKLLP

SEQ ID NO: 10 - protein, 26 aa, synthetic construct
RRKRGSGATNFSLLKQAGDVEENPGP

SEQ ID NO: 11 - RNA, 2820 nt, synthetic construct
  source 1..2820: mol_type = other RNA
atggacggaactctgttccccggcgacgacgatctggccatccccgccaccgagtttttcagcacaaaggccgctaagaagcctgaagccaagagagaggccatcgtgaaggccgatgaagatgataacgaggaaaccctgaagcagagactgacaaatctcgaaaagaagatcaccaacgtgaccacaaagttcgagcaaattgaaaagtgctgcaagcggaacgatgaagttctattccggctggaaaaccacgccgagaccctgagagccgccatgatcagcctggccaagaaaatcgacgtgcaaacaggcagacggccttacgaaagaagaaagaggggcagcggcgccacaaacttctcactgctgaaacaggccggcgacgtggaggaaaatcctggccctatgggcgccgccgctagcatccagacaacagtgaacaccctgagcgagcggatcagcagcaagctggagcaggaggccaatgcctctgcccagaccaaatgcgacatcgagatcggcaatttttacatcagacagaaccacgggtgcaacctgaccgtgaagaacatgtgctccgctgacgcggatgcccagctggatgccgtcctgtctgccgccactgaaacctacagcggcctgacaccagaacaaaaggcctacgttcctgccatgttcaccgccgccctgaacatccagacctctgtgaacaccgtggtgcgggacttcgagaattacgtgaagcagacatgcaacagcagcgctgtggtggacaacaagctgaaaattcagaacgtgatcatagacgagtgttacggagcccctggcagccctaccaatctcgagttcatcaacaccggcagcagcaagggcaattgcgccatcaaggccctgatgcagctgaccacgaaggctaccacccagatcgcccctaagcaggtggccggcaccggcgtgcagttctacatgatcgtgatcggcgtgatcattctggccgccctctttatgtactacgccaagcgtatgctgttcaccagcactaatgataagatcaagctgattctggctaataaagagaacgtgcactggaccacatatatggacaccttctttagaaccagccccatggtgatcgccaccaccgacatgcaaaatagaagaaagaggggctctggagccaccaactttagcctgctgaagcaggctggagatgtggaagagaaccctggccctatgatgacacctgagaacgatgaggagcaaacctctgtgttcagcgccacggtgtacggcgataagatccagggtaagaataagcggaagagagtgatcggcctgtgtatccggatctctatggtgatcagcttgctgagcatgatcaccatgtctgccttcctgatcgtgcggctgaaccagtgcatgtccgccaacgaagccgccatcaccgacgccgcagtggccgtggccgcagctagcagcacccatagaaaagtggcaagcagcacaacccagtacgatcacaaagagtcctgtaacggcctgtactaccaaggctcttgctacatcctgcacagcgactatcagctgttctcagacgccaaggctaactgcactgccgaatccagcaccctgcctaacaaaagcgacgtgctgattacctggctgatcgactacgtggaggacacctggggctctgatggcaaccccatcactaaaaccaccagcgattaccaggacagcgacgtcagccaggaggtcagaaaatatttctgcgtgaagaccatgaaccggagaaagagaggcagcggcgccaccaacttctcactgctgaagcaggccggagatgtggaagaaaacccaggcccaatgaagaccatcagcgtggtgacactgctgtgcgtgctccccgctgttgtgtactccacctgtacagtgcctacaatgaacaacgccaagctgacaagcaccgagaccagcttcaacgataagcagaaggtgaccttcacctgtgaccaaggataccacagcctggaccctaacgctgtgtgcgagacagacaagtggaagtacgagaacccttgtaaaaagatgtgtacagtgtctgactacgtgagcgagctgtacgacaagcccctgtacgaggtcaattccaccatgaccctgagctgtaacggcgagacaaagtacttcagatgcgaggaaaagaacggcaacacaagctggaacgacaccgtgacttgccccaacgccgaatgccagccgctgcagctggagcacggcagctgccagcctgtcaaagagaagtatagcttcggtgaatacattacaatcaattgcgacgtgggctacgaggtgatcggcgccagctacatcagctgtaccgctaatagctggaacgtcatcccttcctgccagcagaagtgcgacatgccttctctgtctaacggcctgatctccggatcaacattttccatcggcggcgtgatccacctgagctgcaagtccggcttcatcctgaccggctccccttctagcacctgcatcgatggcaagtggaaccccatccttccaacctgcgtgagatccaacaaggagttcgaccccgtggacgacggccccgacgacgagacagacctcagcaagctgagcaaggatgtggtccagtacgagcaggaaatcgagagcctagaggccacctaccacatcatcatcgtggctctgacaatcatgggagtgatcttcctgatctctgttatcgtgctggtgtgctcctgtgacaaaaacaacgaccagtacaagttccacaagctgctgcct

SEQ ID NO: 12 - RNA, 43 nt, synthetic construct
  source 1..43: mol_type = other RNA
gagactcttctggtccccacagactcagagagaacccgccacc

SEQ ID NO: 13 - RNA, 110 nt, synthetic construct
  source 1..110: mol_type = other RNA
gctggagcctcggtggccatgcttcttgccccttgggcctccccccagcccctcctccccttcctgcacccgtacccccgtggtctttgaataaagtctgagtgggcggc

SEQ ID NO: 14 - RNA, 121 nt, synthetic construct
  source 1..121: mol_type = other RNA
aaaaaaaaaaaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaa